SEQUENCE LISTING (11 sequences)

SEQ ID NO: 1 - protein, 311 aa, Homo sapiens
MEATRRRQHLGATGGPGAQLGASFLQARHGSVSADEAARTAPFHLDLWFYFTLQNWVLDFGRPIAMLVFPLEWFPLNKPSVGDYFHMAYNVITPFLLLKLIERSPRTLPRSITYVSIIIFIMGASIHLVGDSVNHRLLFSGYQHHLSVRENPIIKNLKPETLIDSFELLYYYDEYLGHCMWYIPFFLILFMYFSGCFTASKAESLIPGPALLLVAPSGLYYWYLVTEGQIFILFIFTFFAMLALVLHQKRKRLFLDSNGLFLFSSFALTLLLVALWVAWLWNDPVLRKKYPGVIYVPEPWAFYTLHVSSRH

SEQ ID NO: 2 - DNA, 936 bp, Homo sapiens
atggaggcgacgcggaggcggcagcacctgggagcgacgggcggcccaggcgcgcagctgggcgcctccttcctgcaggccaggcatggctctgtgagcgctgatgaggctgcccgcacggctcccttccacctcgacctctggttctacttcacactgcagaactgggttctggactttgggcgtcccattgccatgctggtattccctctcgagtggtttccactcaacaagcccagtgttggggactacttccacatggcctacaacgtcatcacgccctttctcttgctcaagctcatcgagcggtccccccgcaccctgccacgctccatcacgtacgtgagcatcatcatcttcatcatgggtgccagcatccacctggtgggtgactctgtcaaccaccgcctgctcttcagtggctaccagcaccacctgtctgtccgtgagaaccccatcatcaagaatctcaagccggagacgctgatcgactcctttgagctgctctactattatgatgagtacctgggtcactgcatgtggtacatccccttcttcctcatcctcttcatgtacttcagcggctgctttactgcctctaaagctgagagcttgattccagggcctgccctgctcctggtggcacccagtggcctgtactactggtacctggtcaccgagggccagatcttcatcctcttcatcttcaccttcttcgccatgctggccctcgtcctgcaccagaagcgcaagcgcctcttcctggacagcaacggcctcttcctcttctcctccttcgcactgaccctcttgcttgtggcgctctgggtcgcctggctgtggaatgaccctgttctcaggaagaagtacccgggtgtcatctacgtccctgagccctgggctttctacacccttcacgtcagcagtcggcactga

SEQ ID NO: 3 - DNA, 270 bp, Artificial Seqeunce
  Synthetic Polynucleotide
ccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 4 - DNA, 2343 bp, Artificial Sequence
  Synthetic Polynucleotide
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcttaagggccgaggcggccagatctttcgaagatatcggcgccgctagcgcggccgcatggaggcgacgcggaggcggcagcacctgggagcgacgggcggcccaggcgcgcagctgggcgcctccttcctgcaggccaggcatggctctgtgagcgctgatgaggctgcccgcacggctcccttccacctcgacctctggttctacttcacactgcagaactgggttctggactttgggcgtcccattgccatgctggtattccctctcgagtggtttccactcaacaagcccagtgttggggactacttccacatggcctacaacgtcatcacgccctttctcttgctcaagctcatcgagcggtccccccgcaccctgccacgctccatcacgtacgtgagcatcatcatcttcatcatgggtgccagcatccacctggtgggtgactctgtcaaccaccgcctgctcttcagtggctaccagcaccacctgtctgtccgtgagaaccccatcatcaagaatctcaagccggagacgctgatcgactcctttgagctgctctactattatgatgagtacctgggtcactgcatgtggtacatccccttcttcctcatcctcttcatgtacttcagcggctgctttactgcctctaaagctgagagcttgattccagggcctgccctgctcctggtggcacccagtggcctgtactactggtacctggtcaccgagggccagatcttcatcctcttcatcttcaccttcttcgccatgctggccctcgtcctgcaccagaagcgcaagcgcctcttcctggacagcaacggcctcttcctcttctcctccttcgcactgaccctcttgcttgtggcgctctgggtcgcctggctgtggaatgaccctgttctcaggaagaagtacccgggtgtcatctacgtccctgagccctgggctttctacacccttcacgtcagcagtcggcactgagtctctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 5 - DNA, 6028 bp, Adeno Associated Virus
ctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcttaagggccgaggcggccagatctttcgaagatatcggcgccgctagcgcggccgcatggaggcgacgcggaggcggcagcacctgggagcgacgggcggcccaggcgcgcagctgggcgcctccttcctgcaggccaggcatggctctgtgagcgctgatgaggctgcccgcacggctcccttccacctcgacctctggttctacttcacactgcagaactgggttctggactttgggcgtcccattgccatgctggtattccctctcgagtggtttccactcaacaagcccagtgttggggactacttccacatggcctacaacgtcatcacgccctttctcttgctcaagctcatcgagcggtccccccgcaccctgccacgctccatcacgtacgtgagcatcatcatcttcatcatgggtgccagcatccacctggtgggtgactctgtcaaccaccgcctgctcttcagtggctaccagcaccacctgtctgtccgtgagaaccccatcatcaagaatctcaagccggagacgctgatcgactcctttgagctgctctactattatgatgagtacctgggtcactgcatgtggtacatccccttcttcctcatcctcttcatgtacttcagcggctgctttactgcctctaaagctgagagcttgattccagggcctgccctgctcctggtggcacccagtggcctgtactactggtacctggtcaccgagggccagatcttcatcctcttcatcttcaccttcttcgccatgctggccctcgtcctgcaccagaagcgcaagcgcctcttcctggacagcaacggcctcttcctcttctcctccttcgcactgaccctcttgcttgtggcgctctgggtcgcctggctgtggaatgaccctgttctcaggaagaagtacccgggtgtcatctacgtccctgagccctgggctttctacacccttcacgtcagcagtcggcactgagtctctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgct

SEQ ID NO: 6 - DNA, 280 bp, Artificial Sequence
  Synthetic Polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctac

SEQ ID NO: 7 - protein, 736 aa, Adeno Associated Virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - DNA, 141 bp, Adeno Associated Virus
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 9 - DNA, 106 bp, Adeno Associated Virus
ctgcgcgctcgctcgctcactgaggccgcccgggcaaagcccgggcgtcgggcgacctttggtcgcccggcctcagtgagcgagcgagcgcgcagagagggagtgg

SEQ ID NO: 10 - DNA, 147 bp, Artificial Sequence
  Synthetic Polynucleotide
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattc

SEQ ID NO: 11 - DNA, 60 bp, Artificial Sequence
  Synthetic Polynucleotide
gtaagtttagtctttttgtcttttatttcaggtcccggatccggtggtggtgcaaatcaa

(19) United States
(12) Patent Application Publication
(10) Pub. No.: US 2022/0193259 A1
(43) Pub. Date: Jun. 23, 2022

(54) GENE THERAPY FOR TREATING OR PREVENTING VISUAL EFFECTS IN BATTEN DISEASE

(71) Applicants: SANFORD RESEARCH, Sioux Falls, SD (US); RESEARCH INSTITUTE AT NATIONWIDE CHILDREN'S HOSPITAL, Columbus, OH (US)

(72) Inventors: Kathrin Meyer, Columbus, OH (US); Shibi Likhite, Columbus, OH (US); Brian K. Kaspar, Columbus, OH (US); Jill M. Weimer, Sioux Falls, SD (US)

(21) Appl. No.: 17/604,188

(22) PCT Filed: Apr. 15, 2020
(86) PCT No.: PCT/US20/28366

§ 371 Date: Oct. 15, 2021

Related U.S. Application Data
(60) Provisional application No. 62/834,340, filed on Apr. 15, 2019.

(51) Int. Cl. A61K 48/00; C07K 14/705; A61P 27/02; C12N 15/86

(52) U.S. Cl. CPC A61K 48/005; C07K 14/705; A61P 27/02; C12N 2750/14171; A61K 48/0075; C12N 2750/14143; C12N 15/86

(57) ABSTRACT

The present disclosure relates to gene therapy methods of preserving photoreceptors and/or inhibiting or preventing retinal degeneration in Batten disease patients, including recombinant adeno-associated vims (rAAV) delivery of a neuronal ceroid lipofuscinosis neuronal 6 (CLN6) polynucleotide.

### CROSS REFERENCE

This application claims priority to U.S. Provisional Patent Application Ser. No. 62/834,340 filed Apr. 15, 2019, incorporated by reference herein in its entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55599_Seqlisting.txt”, which was created on Apr. 14, 2020 and is 23.994 bytes in size. The subject matter of the Sequence Listing is incorporated herein in its entirety by reference.

### FIELD

The present disclosure relates to gene therapy methods for treating or preventing visual effects in Batten disease patients by delivery of a ceroid lipofuscinosis neuronal 6 (CLN6) encoding polynucleotide.

### BACKGROUND

Neuronal ceroid lipofuscinoses (NCLs) are a group of severe neurodegenerative disorders, which are collectively referred to as Batten disease. These disorders affect the nervous system and typically cause worsening problems with e.g. movement and thinking ability. The different NCLs are distinguished by their genetic cause.

CLN6-Batten disease can occur as two different forms: variant late-infantile (vLINCL), the more common form, and adult onset NCL (also called type A Kufs disease) (Cannelii et al., Biochem Biophys Res Commun. 2009; 379(4):892-7, Arsov et al., Am J Hum Genet. 2011; 88(5):566-73). With vLINCL (referred to here as CLN6-Batten disease), age of onset is between 18 months and six years and death typically occurs by age 12-15. CLN6-Batten disease initially presents as impaired language and delayed motor/cognitive development in early childhood, with most patients being wheelchair-bound within four years of disease onset (Canafoglia et al., Neurology. 2015; 85(4):316-24). The disease progresses to include visual loss, severe motor deficits, recurrent seizures, dementia and other neurodegenerative symptoms.

CLN6 is a 311 amino acid protein with seven predicted transmembrane domains, and is predominately localized to the endoplasmic reticulum. As with other CLN proteins, its exact function remains unclear; however, it has been implicated in intracellular trafficking and lysosomal function. There are currently over 70 characterized disease-causing mutations in CLN6 (Warrier et al., Biochimica et Biophysica Acta. 2013; 1832(11):1827-30) with most of these mutations leading to either a complete loss of CLN6 protein or production of truncated CLN6 protein products that are thought to be highly unstable and/or non-functional. Several naturally-occurring animal models of CLN6-Batten disease have been described; these include sheep, canine and mouse models. The spontaneous mutation found in the Cln6^{nclf} mouse model (referred to herein as “Cln6^{nclf} mice”) recapitulates many of the pathological and behavioral aspects of the disease (Morgan et al., PLoS One. 2013; 8(11):e78694). The Cln6^{nclf} mice contain an insertion of an additional cytosine (c.307insC, frame shift after P102), resulting in a premature stop codon that is homologous to a mutation commonly found in CLN6-Batten disease patients (Gao et al., Am J Hum Genet. 2002; 70(2):324-35, Wheeler et al., Am J Hum Genet. 2002; 70(2):537-42).

Currently, there are no therapies that can prevent or reverse the visual effects of Batten disease. Thus, there is a need in the art for treatments for these effects in Batten disease patients.

### SUMMARY

Provided herein are gene therapy methods for preventing the visual effects of Batten disease in a patient in need thereof by delivery of a CLN6 polynucleotide to a subject using a gene delivery vector to, for example, preserve the photoreceptors, prevent or inhibit the degeneration of the photoreceptors, and/or inhibit the degeneration of the retina.

Provided herein are methods of preserving photoreceptors in an individual with Batten disease in need thereof, comprising administering to the individual a pharmaceutical composition comprising a therapeutically effective amount of a gene therapy vector encoding a CLN6 polypeptide. In addition, provided are compositions for preserving photoreceptors in an individual with Batten disease in need thereof, wherein the composition comprises a therapeutically effective amount of a gene therapy vector encoding a CLN6 polypeptide. Also provided are uses of a therapeutically effective amount of a gene therapy vector encoding a CLN6 polypeptide for the preparation of a medicament for preserving photoreceptors in an individual with Batten disease in need thereof. In some embodiments, the CLN6 polypeptide is at least 90% identical to a polypeptide of SEQ ID NO:1. In some embodiments, the gene therapy vector is a viral vector. In some embodiments, the viral vector is an adeno-associated virus (AAV), an adenovirus, a retrovirus, a pox virus, a lentivirus, an adenovirus, a vaccinia virus, or a herpes simplex virus. In some embodiments, the viral vector is an AAV. In some embodiments, the AAV is selected from the group consisting of an AAV1, an AAV2, an AAV3, an AAV4, an AAV5, an AAV6, an AAV7, an AAV8, an AAV9, an AAVrhS, an AAVrh10 vector, an AAVrh33, an AAVrh34, an AAVrh74, an AAV Anc80, an AAVPHP.B, and an AAV-DJ. In some embodiments, the AAV is a recombinant AAV9 (rAAV9) comprising an rAAV9 genome comprising, in 5′ to 3′ order: a first inverted repeat, a chicken beta actin (CB) promoter comprising the nucleic acid sequence of SEQ ID NO: 3, a polynucleotide encoding a Ceroid lipofuscinosis neuron protein 6 (CLN6) polypeptide comprising the amino acid sequence 90% identical to SEQ ID NO:1, and a second inverted repeat. In some embodiments, the rAAV9 genome further comprises a cytomegalovirus (CMV) enhancer. In some embodiments, the rAAV9 genome further comprises a SV40 intron. In some embodiments, the rAAV9 genome further comprises a bovine growth hormone polyadenylation poly A sequence. In some embodiments, the rAAV9 genome is a single-stranded genome or a self-complementary genome. In some embodiments, the rAAV9 genome is a self-complementary genome comprising in 5′ to 3′ order: a first AAV inverted terminal repeat, a CMV enhancer, a CB promoter comprising the nucleotide sequence of SEQ ID NO: 3, an SV40 intron, a polynucleotide encoding the CLN6 polypeptide of SEQ ID NO: 1 and a second AAV inverted terminal repeat. In some embodiments, the rAAV9 genome is a self-complementary genome comprising: a first AAV inverted terminal repeat, a CB promoter comprising the sequence of SEQ ID NO: 3, a polynucleotide encoding the CLN6 polypeptide of SEQ ID NO: 1, a bovine growth hormone polyadenylation poly A sequence and a second AAV inverted terminal repeat. In some embodiments, the rAAV9 genome is a self-complementary genome comprising: a first AAV inverted terminal repeat, a CMV enhancer, a CB promoter comprising the sequence of SEQ ID NO: 3, an SV40 intron, a polynucleotide encoding the CLN6 polypeptide of SEQ ID NO: 1, a bovine growth hormone polyadenylation poly A sequence and a second AAV inverted terminal repeat. In some embodiments, the AAV inverted terminal repeats are AAV2 inverted terminal repeats. In some embodiments, the pharmaceutical composition is administered intracerebroventricularly, intrathecally, intraperenchymally, intravenously, subretinally, intraocularly, intravitreally, or a combination thereof. Intrathecal delivery refers to delivery into the space under the arachnoid membrane of the brain or spinal cord. In some embodiments, intrathecal administration is via intracisternal injection or intralumbar injection. In some embodiments, the pharmaceutical composition is administered intracerebroventricularly. In some embodiments, wherein about 1×10⁸ vg to about 1×10¹⁵ vg of the rAAV viral particle is administered per gram body weight of the individual. In some embodiments, symptoms of visual failure are prevented or ameliorated. In some embodiments, photoreceptor cells in the central retina of the individual are substantially preserved. In some embodiments, the individual comprises a retina comprising at least 4 layers of photoreceptor cells 6 months after the treatment. In some embodiments, wherein the individual comprises a retina comprising at least 8 layers of photoreceptor cells at least 6 months after the treatment. In some embodiments, the individual comprises a retina comprising at least 4 layers of photoreceptor cells at least 9 months after the treatment. In some embodiments, the individual comprises a retina comprising at least 8 layers of photoreceptor cells at least 9 months after the treatment. In some embodiments, the individual is a less than 10 years old. In some embodiments, the individual is less than 1 year old. In additional embodiments, wherein the individual retained more than 4 layers of retina at least 6 months after the treatment. In additional embodiments, wherein the individual retained more than 4 layers of photoreceptor cells at least 9 months after the treatment. In additional embodiments, wherein the individual retained more than 8 layers of photoreceptor cells at least 9 months after the treatment. In some embodiments, the individual comprises a CLN6 gene comprising a mutation related to Batten disease. In some embodiments, the method further comprises detecting a mutation related to Batten disease in a CLN6 gene of the individual. In some embodiments, the pharmaceutical composition further comprises a pharmaceutically acceptable excipient, carrier, or diluent. In some embodiments, the excipient comprises a non-ionic, low-osmolar compound, a buffer, a polymer, a salt, or a combination thereof. In some embodiments, the non-ionic, low-osmolar contrast agent is selected from the group consisting of iobitridol, iohexol, iomeprol, iopamidol, iopentol, iopromide, ioversol, ioxilan, and combinations thereof.

Provided herein are methods of inhibiting retinal degeneration in an individual with Batten disease in need thereof, comprising administering to the individual a pharmaceutical composition comprising a therapeutically effective amount of a gene therapy vector encoding a CLN6 polypeptide. In addition, provided are compositions for inhibiting retinal degeneration in an individual with Batten disease in need thereof, wherein the composition comprises a therapeutically effective amount of a gene therapy vector encoding a CLN6 polypeptide. Also provided are uses of a therapeutically effective amount of a gene therapy vector encoding a CLN6 polypeptide for the preparation of a medicament for inhibiting retinal degeneration in an individual with Batten disease in need thereof. In some embodiments, the CLN6 polypeptide is at least 90% identical to a polypeptide of SEQ ID NO:1. In some embodiments, the gene therapy vector is a viral vector. In some embodiments, the viral vector is an adeno-associated virus (AAV), an adenovirus, a retrovirus, a pox virus, a lentivirus, an adenovirus, a vaccinia virus, or a herpes simplex virus. In some embodiments, the viral vector is an AAV. In some embodiments, the AAV is selected from the group consisting of an AAV1, an AAV2, an AAV3, an AAV4, an AAV5, an AAV6, an AAV7, an AAV8, an AAV9, an AAVrhS, an AAVrh10 vector, an AAVrh33, an AAVrh34, an AAVrh74, an AAV Anc80, an AAVPHP.B, and an AAV-DJ. In some embodiments, the AAV is a recombinant AAV9 (rAAV9) comprising an rAAV9 genome comprising, in 5′ to 3′ order: a first inverted repeat, a chicken beta actin (CB) promoter comprising the nucleic acid sequence of SEQ ID NO: 3, a polynucleotide encoding a Ceroid lipofuscinosis neuron protein 6 (CLN6) polypeptide comprising the amino acid sequence 90% identical to SEQ ID NO:1, and a second inverted repeat. In some embodiments, the rAAV9 genome further comprises a cytomegalovirus (CMV) enhancer. In some embodiments, the rAAV9 genome further comprises a SV40 intron. In some embodiments, the rAAV9 genome further comprises a bovine growth hormone polyadenylation poly A sequence. In some embodiments, the rAAV9 genome is a single-stranded genome or a self-complementary genome. In some embodiments, the rAAV9 genome is a self-complementary genome comprising in 5′ to 3′ order: a first AAV inverted terminal repeat, a CMV enhancer, a CB promoter comprising the nucleotide sequence of SEQ ID NO: 3, an SV40 intron, a polynucleotide encoding the CLN6 polypeptide of SEQ ID NO: 1 and a second AAV inverted terminal repeat. In some embodiments, the rAAV9 genome is a self-complementary genome comprising: a first AAV inverted terminal repeat, a CB promoter comprising the sequence of SEQ ID NO: 3, a polynucleotide encoding the CLN6 polypeptide of SEQ ID NO: 1, a bovine growth hormone polyadenylation poly A sequence and a second AAV inverted terminal repeat. In some embodiments, the rAAV9 genome is a self-complementary genome comprising: a first AAV inverted terminal repeat, a CMV enhancer, a CB promoter comprising the sequence of SEQ ID NO: 3, an SV40 intron, a polynucleotide encoding the CLN6 polypeptide of SEQ ID NO: 1, a bovine growth hormone polyadenylation poly A sequence and a second AAV inverted terminal repeat. In some embodiments, the AAV inverted terminal repeats are AAV2 inverted terminal repeats. In some embodiments, the pharmaceutical composition is administered intracerebroventricularly, intrathecally, intraperenchymally, intravenously, subretinally, intraocularly, intravitreally, or a combination thereof. Intrathecal delivery refers to delivery into the space under the arachnoid membrane of the brain or spinal cord. In some embodiments, intrathecal administration is via intracisternal injection or intralumbar injection. In some embodiments, the pharmaceutical composition is administered intracerebroventricularly. In some embodiments, wherein about 1×10⁸ vg to about 1×10¹⁵ vg of the rAAV viral particle is administered per gram body weight of the individual. In some embodiments, symptoms of visual failure are prevented or ameliorated. In some embodiments, photoreceptor cells in the central retina of the individual are substantially preserved. In some embodiments, the individual comprises a retina comprising at least 4 layers of photoreceptor cells 6 months after the treatment. In some embodiments, wherein the individual comprises a retina comprising at least 8 layers of photoreceptor cells at least 6 months after the treatment. In some embodiments, the individual comprises a retina comprising at least 4 layers of photoreceptor cells at least 9 months after the treatment. In some embodiments, the individual comprises a retina comprising at least 8 layers of photoreceptor cells at least 9 months after the treatment. In additional embodiments, wherein the individual retained more than 4 layers of retina at least 6 months after the treatment. In additional embodiments, wherein the individual retained more than 4 layers of photoreceptor cells at least 9 months after the treatment. In additional embodiments, wherein the individual retained more than 8 layers of photoreceptor cells at least 9 months after the treatment. In some embodiments, the individual is a less than 10 years old. In some embodiments, the individual is less than 1 year old. In some embodiments, the individual comprises a CLN6 gene comprising a mutation related to Batten disease. In some embodiments, the method further comprises detecting a mutation related to Batten disease in a CLN6 gene of the individual. In some embodiments, the pharmaceutical composition further comprises a pharmaceutically acceptable excipient, carrier, or diluent. In some embodiments, the excipient comprises a non-ionic, low-osmolar compound, a buffer, a polymer, a salt, or a combination thereof. In some embodiments, the non-ionic, low-osmolar contrast agent is selected from the group consisting of iobitridol, iohexol, iomeprol, iopamidol, iopentol, iopromide, ioversol, ioxilan, and combinations thereof.

Provided herein are methods of treating the visual effects of an individual with Batten disease in need thereof, comprising administering to the individual a pharmaceutical composition comprising a therapeutically effective amount of a gene therapy vector encoding a CLN6 polypeptide. In addition, provided are compositions for treating the visual effects of an individual with Batten disease in need thereof, wherein the composition comprises a therapeutically effective amount of a gene therapy vector encoding a CLN6 polypeptide. Also provided are uses of a therapeutically effective amount of a gene therapy vector encoding a CLN6 polypeptide for the preparation of a medicament for treating the visual effects of an individual with Batten disease in need thereof. In some embodiments, the CLN6 polypeptide is at least 90% identical to a polypeptide of SEQ ID NO:1. In some embodiments, the gene therapy vector is a viral vector. In some embodiments, the viral vector is an adeno-associated virus (AAV), an adenovirus, a retrovirus, a pox virus, a lentivirus, an adenovirus, a vaccinia virus, or a herpes simplex virus. In some embodiments, the viral vector is an AAV. In some embodiments, the AAV is selected from the group consisting of an AAV1, an AAV2, an AAV3, an AAV4, an AAV5, an AAV6, an AAV7, an AAV8, an AAV9, an AAVrhS, an AAVrh10 vector, an AAVrh33, an AAVrh34, an AAVrh74, an AAV Anc80, an AAVPHP.B, and an AAV-DJ. In some embodiments, the AAV is a recombinant AAV9 (rAAV9) comprising an rAAV9 genome comprising, in 5′ to 3′ order: a first inverted repeat, a chicken beta actin (CB) promoter comprising the nucleic acid sequence of SEQ ID NO: 3, a polynucleotide encoding a Ceroid lipofuscinosis neuron protein 6 (CLN6) polypeptide comprising the amino acid sequence 90% identical to SEQ ID NO:1, and a second inverted repeat. In some embodiments, the rAAV9 genome further comprises a cytomegalovirus (CMV) enhancer. In some embodiments, the rAAV9 genome further comprises a SV40 intron. In some embodiments, the rAAV9 genome further comprises a bovine growth hormone polyadenylation poly A sequence. In some embodiments, the rAAV9 genome is a single-stranded genome or a self-complementary genome. In some embodiments, the rAAV9 genome is a self-complementary genome comprising in 5′ to 3′ order: a first AAV inverted terminal repeat, a CMV enhancer, a CB promoter comprising the nucleotide sequence of SEQ ID NO: 3, an SV40 intron, a polynucleotide encoding the CLN6 polypeptide of SEQ ID NO: 1 and a second AAV inverted terminal repeat. In some embodiments, the rAAV9 genome is a self-complementary genome comprising: a first AAV inverted terminal repeat, a CB promoter comprising the sequence of SEQ ID NO: 3, a polynucleotide encoding the CLN6 polypeptide of SEQ ID NO: 1, a bovine growth hormone polyadenylation poly A sequence and a second AAV inverted terminal repeat. In some embodiments, the rAAV9 genome is a self-complementary genome comprising: a first AAV inverted terminal repeat, a CMV enhancer, a CB promoter comprising the sequence of SEQ ID NO: 3, an SV40 intron, a polynucleotide encoding the CLN6 polypeptide of SEQ ID NO: 1, a bovine growth hormone polyadenylation poly A sequence and a second AAV inverted terminal repeat. In some embodiments, the AAV inverted terminal repeats are AAV2 inverted terminal repeats. In some embodiments, the pharmaceutical composition is administered intracerebroventricularly, intrathecally, intraperenchymally, intravenously, subretinally, intraocularly, intravitreally, or a combination thereof. Intrathecal delivery refers to delivery into the space under the arachnoid membrane of the brain or spinal cord. In some embodiments, intrathecal administration is via intracisternal injection or intralumbar injection. In some embodiments, the pharmaceutical composition is administered intracerebroventricularly. In some embodiments, wherein about 1×10⁸ vg to about 1×10¹⁵ vg of the rAAV viral particle is administered per gram body weight of the individual. In some embodiments, symptoms of visual failure are prevented or ameliorated. In some embodiments, photoreceptor cells in the central retina of the individual are substantially preserved. In some embodiments, the individual comprises a retina comprising at least 4 layers of photoreceptor cells 6 months after the treatment. In some embodiments, wherein the individual comprises a retina comprising at least 8 layers of photoreceptor cells at least 6 months after the treatment. In some embodiments, the individual comprises a retina comprising at least 4 layers of photoreceptor cells at least 9 months after the treatment. In some embodiments, the individual comprises a retina comprising at least 8 layers of photoreceptor cells at least 9 months after the treatment. In additional embodiments, wherein the individual retained more than 4 layers of retina at least 6 months after the treatment. In additional embodiments, wherein the individual retained more than 4 layers of photoreceptor cells at least 9 months after the treatment. In additional embodiments, wherein the individual retained more than 8 layers of photoreceptor cells at least 9 months after the treatment. In some embodiments, the individual is a less than 10 years old. In some embodiments, the individual is less than 1 year old. In some embodiments, the individual comprises a CLN6 gene comprising a mutation related to Batten disease. In some embodiments, the method further comprises detecting a mutation related to Batten disease in a CLN6 gene of the individual. In some embodiments, the pharmaceutical composition further comprises a pharmaceutically acceptable excipient, carrier, or diluent. In some embodiments, the excipient comprises a non-ionic, low-osmolar compound, a buffer, a polymer, a salt, or a combination thereof. In some embodiments, the non-ionic, low-osmolar contrast agent is selected from the group consisting of iobitridol, iohexol, iomeprol, iopamidol, iopentol, iopromide, ioversol, ioxilan, and combinations thereof.

The headings herein are for the convenience of the reader and not intended to be limiting.

The use of ‘may’ and ‘can’ herein is to describe the various embodiments that are included within the claims, and not to indicate uncertainty about the scope of the claims.

### BRIEF DESCRIPTION OF THE DRAWINGS

FIG. 1 provides a schematic of the scAAV genome of scAAV.CB.CLN6.

FIG. 2 provides the nucleic acid sequence of scAAV9.CB.CLN6 gene cassette (SEQ ID NO: 4). The AAV2 ITR nucleic acid sequence is in italics (5′ ITR is set out as SEQ ID NO: 9; 3′ ITR is set out as SEQ ID NO: 8), the CMV enhancer nucleic acid sequence (SEQ ID NO: 6) is underlined with a dotted line, the CB promoter nucleic acid sequence (SEQ ID NO: 3) is underlined with a single line, the SV40 intron nucleic acid sequence (SEQ ID NO: 11) is underlined with a double line, the nucleic acid sequence of the human CLN6 cDNA sequence (SEQ ID NO: 2) is in bold, the nucleic acid sequence of the BGH polyA terminator (SEQ ID NO: 10) is underlined with a dashed line.

FIG. 3 provides the nucleic acid sequence of full AAV.CB.CLN6 (SEQ ID NO: 5).

FIG. 4 provides plots of visual acuity of Cln6^{nclf} mice (administered with scAAV9.CB.CLN6 or PBS) and WT mice using OptoMotry optokinetic tracking. A single, postnatal day 1 injection of scAAV9.CB.CLN6 delivered via CSF of partially restores visual acuity in male and female Cln6^{nclf} mice. When linear fits are compared, untreated female Cln6^{nclf} mice have a significantly steeper decline than both wild type and AAV9 treated Cln6^{nclf} mice. Slopes between wild type and AAV9 treated Cln6^{nclf} mice did not significantly differ, and did not differ between male mice. Ordinary two-way ANOVA, Tukey correction. For linear fit, slopes were compared using an ordinary one-way ANOVA, Tukey post hoc. Males: n=11-17 at 3M, n=6-14 at 6M, n=3-11 at 9M. Females: n=11-16 at 3M, n=5-12 at 6M, n=3-6 at 9M. Mean±SEM. *p<0.05, **p<0.01, ***p<0.001, ****p<0.0001.

FIG. 5 provides images and plots of autofluorescent storage material accumulation of Cln6^{nclf} mice (administered with scAAV9.CB.CLN6 or PBS) and WT mice. A single, postnatal day 1 injection of scAAV9.CB.CLN6 delivered via CSF prevents storage material accumulation (ASM) in the dorsal lateral geniculate, primary visual cortex and superior colliculus at 6 months and 9 months of age. Mean±SEM, one-way ANOVA for each time point, Bonferroni correction. **p<0.01, ***p<0.001, ****p<0.0001.

FIG. 6 provides images and plots of microglial activiation of Cln6^{nclf} mice (administered with scAAV9.CB.CLN6 or PBS) and WT mice. A single, postnatal day 1 injection of scAAV9.CB.CLN6 delivered via CSF prevented CD68⁺ microglial activation in the dorsal lateral geniculate, primary visual cortex, superior colliculus at 6 months and 9 months of age. Mean±SEM, one-way ANOVA for each time point, Bonferroni correction. *p<0.05, **p<0.01, ***p<0.001, ****p<0.0001.

FIG. 7 provides images and plots of astrocyte reactivity of Cln6^{nclf} mice (administered with scAAV9.CB.CLN6 or PBS) and WT mice.

FIG. 8 provides plots of neuron counts in areas of the brain of Cln6^{nclf} mice (administered with scAAV9.CB.CLN6 or PBS) and WT mice. A single, postnatal day 1 injection of scAAV9.CB.CLN6 delivered via CSF prevents progressive neuronal loss in the dorsal lateral geniculate (DLG), primary visual cortex (V1), and superior colliculus (SupCol), at 6 months and 9 months of age. Mean±SEM, one-way ANOVA for each time point, Bonferroni correction. **p<0.01, ***p<0.001, ****p<0.0001.

FIG. 9 provides images of retinas and a plot of numbers of photoreceptors of Cln6^{cnlf} mice (administered with scAAV9.CB.CLN6 or PBS) and WT mice. Retinal sections near the optic nerve head stained with cresyl violet show retinal lamination and photoreceptors (blue). A single, postnatal day 1 injection of scAAV9.CB.CLN6 delivered via CSF prevents progressive photoreceptor loss in 3 month, 6 month, and 9 month Cln6^{nclf} mice. Wild type animals show 10 to 12 rows of photoreceptor nuclei, while untreated Cln6^{nclf} mice retain only one layer of photoreceptors by 9 months of age. In contrast, AAV9 treated Cln6^{nclf} mice maintain 8 to 10 rows of photoreceptors at all time point examined. Quantification of photoreceptor density presented in the plot.

FIG. 10 provides images of the CLN6 distribution in the layers of the retina of Cln6^{nclf} mice (administered with scAAV9.CB.CLN6 or PBS). Retinal sections immunolabeled with anti-human CLN6 (red) and rhodopsin (photoreceptors, green) antibodies detected hCLN6 in AAV9 treated Cln6^{nclf} mice at 3, 6 and 9 months of age, primarily in the RGC, INL, ONL and RPE layers. Retinal sections double stained with anti-human CLN6 (red) and anti-glutamine synthetase (Müller glia, green), showed no colocalization, indicating scAAV9.CB.CLN6 preferentially targeted nuclear and epithelial layers over glial layers using this dosing strategy. n=6/treatment for each time point, represented by equal numbers of males and females.

### DETAILED DESCRIPTION

The present disclosure provides methods and products for treating or preventing the visual effects of Batten disease in a patient in need thereof, by, for example preserving the photoreceptors, preventing or inhibiting the degeneration of the photoreceptors, and/or inhibiting the degeneration of the retina. The methods involve delivery of a CLN6 polynucleotide to a subject using a gene delivery vector. In some embodiments, the gene therapy vector is an adeno-associated virus (AAV), an adenovirus, a retrovirus, a pox virus, a lentivirus, an adenovirus, a vaccinia virus, or a herpes simplex virus. In some embodiments, the gene delivery vector is an AAV.

As used herein, the terms “vector” or “gene therapy vector”, used interchangeably herein, refers to gene therapy delivery vehicles, or carriers, that deliver therapeutic genes (e.g., encoding therapeutic proteins) to cells. A gene therapy vector is any vector suitable for use in gene therapy, e.g., any vector suitable for the therapeutic delivery of nucleic acid polymers (encoding a polypeptide or a variant thereof) into target cells (e.g., neurons) of a patient. The vector may be of any type, for example it may be a plasmid vector or a minicircle DNA. Typically, the vector is a viral vector. Vectors include both genetically disabled viruses such as adenovirus and nonviral vectors such as liposomes. The viral vector may for example be derived from an adeno-associated virus (AAV), a retrovirus, a pox virus, a vaccinia virus, a lentivirus, a herpes simplex virus, or an adenovirus. For AAV derived vectors, the vector may comprise an AAV genome or a derivative thereof.

Adeno-associated virus (AAV) is a replication-deficient parvovirus, the single-stranded DNA genome of which is about 4.7 kb in length including two 145 nucleotide inverted terminal repeats (ITRs) and may be used to refer to the virus itself or derivatives thereof. The term covers all subtypes and both naturally occurring and recombinant forms, except where specified otherwise. There are multiple serotypes of AAV. The serotypes of AAV are each associated with a specific Glade, the members of which share serologic and functional similarities. Thus, AAVs may also be referred to by the Glade. For example, AAV9 sequences are referred to as “Glade F” sequences (Gao et al., J. Virol., 78: 6381-6388 (2004). The present disclosure contemplates the use of any sequence within a specific Glade, e.g., Glade F. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B 1 genome is provided in Choudhury et al., Mol. Ther., 24(7): 1247-1257 (2016). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992). In some embodiments, the AAV is selected from the group consisting of an AAV1, an AAV2, an AAV3, an AAV4, an AAV5, an AAV6, an AAV7, an AAV8, an AAV9, an AAVrhS, an AAVrh10 vector, an AAVrh33, an AAVrh34, an AAVrh74, an AAV Anc80, an AAVPHP.B, and an AAV-DJ.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The native AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. In some instances, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

The term “AAV” as used herein refers to the wild type AAV virus or viral particles. The terms “AAV,” “AAV virus,” and “AAV viral particle” are used interchangeably herein. The term “rAAV” refers to a recombinant AAV virus or recombinant infectious, encapsulated viral particles. The terms “rAAV,” “rAAV virus,” and “rAAV viral particle” are used interchangeably herein.

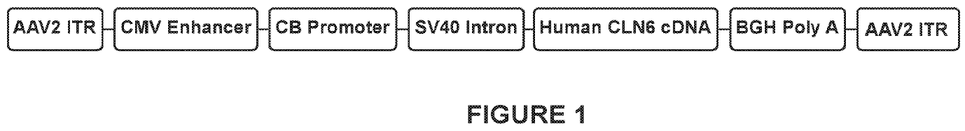

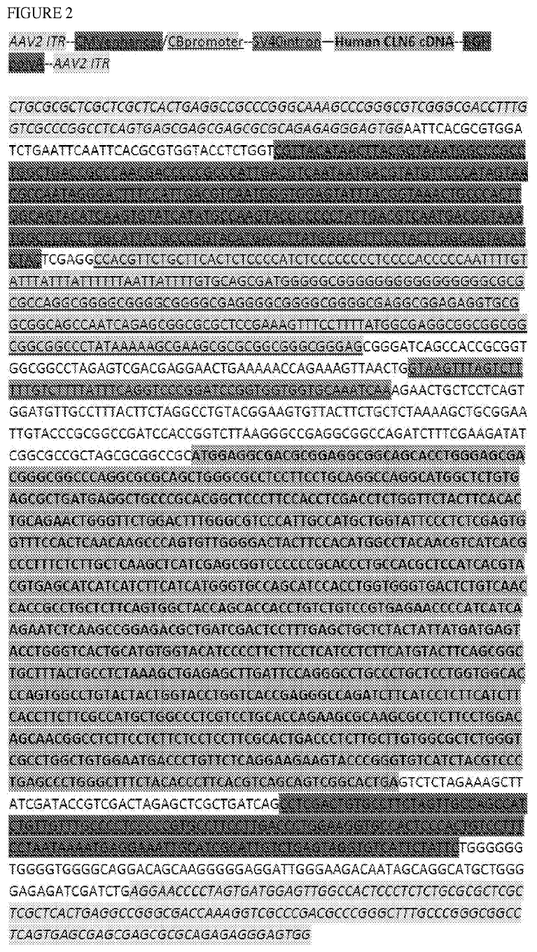

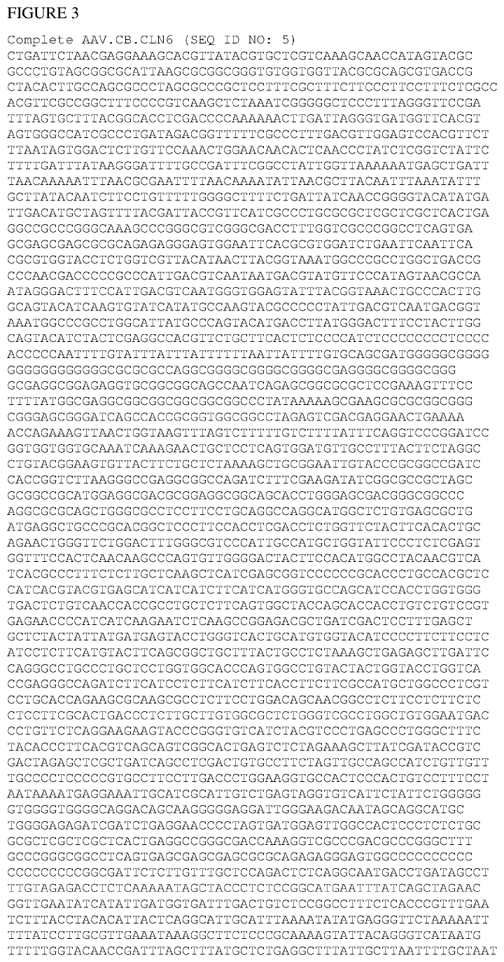

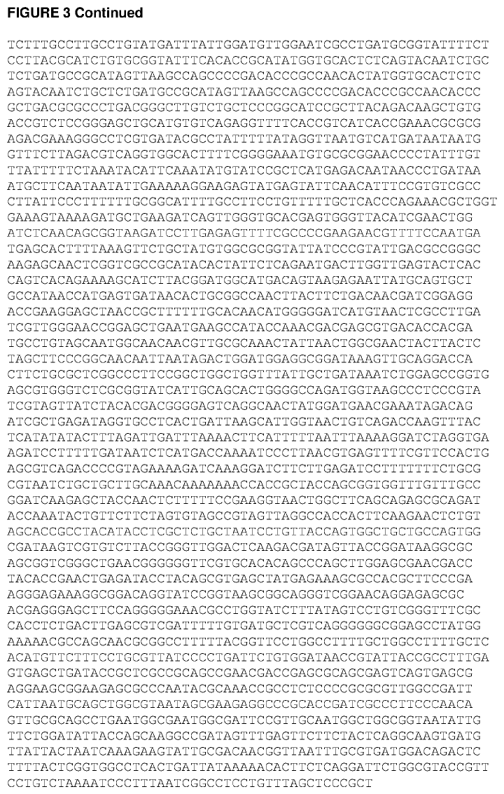

The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest (e.g., a CLN6-encoding polynucleotide) flanked on the 5′ and 3′ ends by inverted terminal repeat (ITR). In some embodiments, the rAAV genome comprises a “gene cassette.” An exemplary gene cassette is set out in FIG. 1A and the nucleic acid sequence of SEQ ID NO: 4. The rAAV genome can be a self-complementary (sc) genome, which is referred to herein as “scAAV genome.” Alternatively, the rAAV genome can be a single-stranded (ss) genome, which is referred to herein as “ssAAV genome.”

The term “scAAV” refers to a rAAV virus or rAAV viral particle comprising a self-complementary genome. The term “ssAAV” refers to a rAAV virus or rAAV viral particle comprising a single-stranded genome.

rAAV genomes provided herein may comprise a polynucleotide encoding a CLN6 polypeptide. CLN6 polypeptides comprise the amino acid sequence set out in SEQ ID NO: 1, or a polypeptide with an amino acid sequence that is at least: 90%, 91%, 92%, 93%, 94%, 95%, 96%, 97%, 98%, or 99% identical to the amino acid sequence set forth in SEQ ID NO: 1, and which encodes a polypeptide with CLN6 activity (e.g., at least one of inhibiting or preventing degeneration of photoreceptors, inhibiting retinal degradation, increasing clearance of lysosomal auto fluorescent storage material, reducing lysosomal accumulation of ATP synthase subunit C, and reducing activation of astrocytes and microglia in a patient when treated as compared to, e.g. the patient prior to treatment).

rAAV genomes provided herein, in some cases, comprise a polynucleotide encoding a CLN6 polypeptide wherein the polynucleotide has the nucleotide sequence set out in SEQ ID NO: 2, or a polynucleotide at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence set forth in SEQ ID NO: 2 and encodes a polypeptide with CLN6 activity (e.g., at least one of inhibiting or preventing degeneration of photoreceptors, inhibiting retinal degeneration, increasing clearance of lysosomal auto fluorescent storage material, reducing lysosomal accumulation of ATP synthase subunit C, and reducing activation of astrocytes and microglia in a patient when treated as compared to, e.g. the patient prior to treatment).

rAAV genomes provided herein, in some embodiments, comprise a polynucleotide sequence that encodes a polypeptide with CLN6 activity and that hybridizes under stringent conditions to the nucleic acid sequence of SEQ ID NO: 2, or the complement thereof. The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing include but are not limited to 0.015 M sodium chloride, 0.0015 M sodium citrate at 65-68° C. or 0.015 M sodium chloride, 0.0015M sodium citrate, and 50% formamide at 42° C. See, for example, Sambrook et al., Molecular Cloning: A Laboratory Manual, 2nd Ed., Cold Spring Harbor Laboratory, (Cold Spring Harbor, N.Y. 1989).

The rAAV genomes provided herein, in some embodiments, comprise one or more AAV ITRs flanking the polynucleotide encoding a CLN6 polypeptide. The CLN6 polynucleotide is operatively linked to transcriptional control elements (including, but not limited to, promoters, enhancers and/or polyadenylation signal sequences) that are functional in target cells to form a gene cassette. Examples of promoters are the chicken β actin promoter and the P546 promoter. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-la promoter, the hemoglobin promoter, and the creatine kinase promoter. Additionally provided herein are a CB promoter sequence set out in SEQ ID NO: 3, and promoter sequences at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence set forth in SEQ ID NO: 3 that are promoters with CB transcription promoting activity. Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a CLN6 RNA transcript when expressed in mammalian cells. One example of such an intron is the SV40 intron.

In some embodiments, there are recombinant adeno-associated virus 9 (rAAV9) encoding a CLN6 polypeptide, comprising an rAAV9 genome comprising in 5′ to 3′ order: a hybrid chicken β-actin (CB) promoter and a polynucleotide encoding the CLN6 polypeptide. In some cases, the rAAV9 genome comprises a self-complementary genome. Alternatively, the rAAV9 genome comprises a single-stranded genome.

Self-complementary recombinant adeno-associated virus 9 (scAAV9) are provided encoding the CLN6 polypeptide set out in SEQ ID NO: 1, in which the genome of the scAAV9 comprises in 5′ to 3′ order: a first AAV inverted terminal repeat, a hybrid chicken β-actin (CB) promoter comprising the sequence of SEQ ID NO: 3, a polynucleotide encoding the CLN6 polypeptide set out in SEQ ID NO: 2 and a second AAV inverted terminal repeat. The polynucleotide encoding the CLN6 polypeptide may be at least 90% identical to SEQ ID NO: 2.

Also provided are scAAV9 with a genome comprising in 5′ to 3′ order: a first AAV inverted terminal repeat, a CMV enhancer, a hybrid chicken β-Actin promoter (cb), an SV40 intron, a polynucleotide encoding the CLN6 polypeptide of SEQ ID NO: 1 and a second AAV inverted terminal repeat; scAAV9 with a genome comprising in 5′ to 3′ order: a first AAV inverted terminal repeat, a CB promoter comprising the sequence of SEQ ID NO: 3, a polynucleotide encoding the CLN6 polypeptide of SEQ ID NO: 1, a bovine growth hormone polyadenylation poly A sequence and a second AAV inverted terminal repeat; and scAAV9 with a genome comprising the gene cassette set out in the nucleic acid sequence of SEQ ID NO: 4.

Also provided are ssAAV9 with a genome comprising in 5′ to 3′ order: a first AAV inverted terminal repeat, a CMV enhancer, a hybrid chicken β-Actin promoter (CB), an SV40 intron, a polynucleotide encoding the CLN6 polypeptide of SEQ ID NO: 1 and a second AAV inverted terminal repeat; ssAAV9 with a genome comprising in 5′ to 3′ order: a first AAV inverted terminal repeat, a CB promoter comprising the sequence of SEQ ID NO: 3, a polynucleotide encoding the CLN6 polypeptide of SEQ ID NO: 1, a bovine growth hormone polyadenylation poly A sequence and a second AAV inverted terminal repeat; or ssAAV9 with a genome comprising the gene cassette set out in the nucleic acid sequence of SEQ ID NO: 4.

The nucleic acid sequence set out in SEQ ID NO: 4 is the gene cassette that is provided in FIG. 1. Provided are rAAV9 comprising an scAAV9 genome or a ssAAV9 genome comprising a nucleic acid sequence that is at least 90% identical to the nucleic acid sequence of SEQ ID NO: 4, at least 95% identical to the nucleic acid sequence of SEQ ID NO: 4, or at least 98% identical to the nucleic acid sequence of SEQ ID NO: 4.

Further provided are nucleic acid molecules comprising a first AAV inverted terminal repeat, a CB promoter comprising the nucleic acid sequence of SEQ ID NO: 3, a nucleic acid sequence encoding the CLN6 polypeptide of SEQ ID NO: 1 and a second AAV inverted terminal repeat. In some embodiments, the polynucleotide encoding the CLN6 polypeptide may be at least 90% identical to the nucleic acid sequence of SEQ ID NO: 2.

Also provided are nucleic acid molecules comprising a first AAV inverted terminal repeat, a CB promoter comprising the nucleotide sequence of SEQ ID NO: 3, an SV40 intron, a nucleic acid sequence encoding the CLN6 polypeptide of SEQ ID NO: 1 and a second AAV inverted terminal repeat. In addition, provided are nucleic acid molecules comprising a first AAV inverted terminal repeat, a CB promoter comprising the nucleotide sequence of SEQ ID NO: 3, a nucleic acid encoding the CLN6 polypeptide of SEQ ID NO: 1, a BGH poly-A sequence and a second AAV inverted terminal repeat. In any of the polynucleotides provided, the CLN6 polypeptide can be encoded by a nucleic acid sequence at least 90% identical to the nucleic acid sequence of SEQ ID NO: 2.

Provided are rAAV with an scAAV genome or an ssAAV genome, wherein the genome comprises a nucleic acid sequence that is at least 90% identical to the nucleic acid sequence of SEQ ID NO: 4, or at least 95% identical to the nucleic acid sequence of SEQ ID NO: 4, or at least 98% identical to the nucleic acid sequence of SEQ ID NO: 4.

The provided rAAV can comprise any of the polynucleotides disclosed herein. In addition, viral particles comprising any of the disclosed nucleic acid s are provided. The rAAV with self-complementary or single stranded genomes are also provided.

Also provided are recombinant adeno-associated virus 9 (rAAV9) viral particles encoding a CLN6 polypeptide, comprising an rAAV9 genome comprising in 5′ to 3′ order: a CMV enhancer comprising a nucleic acid sequence at least 90% identical to SEQ ID NO: 6, a CB promoter comprising a nucleic acid sequence at least 90% identical to SEQ ID NO: 3, and a polynucleotide encoding a CLN6 polypeptide at least 90% identical to the amino acid sequence of SEQ ID NO: 1. In some embodiments, the rAAV9 viral particles provided comprise a self-complementary genome. Alternatively, the rAAV9 viral particles provided comprise a single stranded genome.

Further provided are rAAV9 viral particles, wherein the rAAV9 genome comprises in 5′ to 3′ order: a first AAV inverted terminal repeat, the CMV enhancer comprising a nucleic acid sequence at least 90% identical to SEQ ID NO: 6, the CB promoter comprising a nucleic acid sequence at least 90% identical to SEQ ID NO: 3, the polynucleotide encoding a CLN6 polypeptide at least 90% identical to the amino acid sequence of SEQ ID NO: 1, and a second AAV inverted terminal repeat. The rAAV9 particles provided comprise a polynucleotide encoding the CLN6 polypeptide comprising an amino acid sequence at least 90% identical to SEQ ID NO: 1. Any of the rAAV9 viral particles optionally further comprise an SV40 intron, and/or a BGH poly-A sequence.

In an additional embodiment, the rAAV9 viral particles comprise an AAV9 genome comprising a nucleic acid sequence at least 90% identical to the nucleic acid sequence of SEQ ID NO: 4, at least 95% identical to nucleic acid sequence of SEQ ID NO: 4, or at least 98% identical to the nucleic acid sequence of SEQ ID NO: 4.

In any of the rAAV, the ssAAV or the scAAV provided, the AAV inverted terminal repeats may be AAV2 inverted terminal repeats.

Also provided are nucleic acid molecules comprising an rAAV9 genome comprising in 5′ to 3′ order: a first AAV inverted terminal repeat, a CMV enhancer comprising a nucleic acid sequence at least 90% identical to SEQ ID NO: 6, a CB promoter comprising a nucleic acid sequence at least 90% identical to SEQ ID NO: 3, and a polynucleotide encoding a CLN6 polypeptide at least 90% identical to the amino acid sequence of SEQ ID NO: 1. The provided nucleic acid molecules comprise a self-complementary genome and/or a single stranded genome.

Further provided are nucleic acid molecules comprising a rAAV9 genome that comprises in 5′ to 3′ order: a first AAV inverted terminal repeat, the CMV enhancer comprising a nucleic acid sequence at least 90% identical to SEQ ID NO: 6, the CB promoter comprising a nucleic acid sequence at least 90% identical to SEQ ID NO: 3, the polynucleotide encoding a CLN6 polypeptide at least 90% identical to the amino acid sequence of SEQ ID NO: 1, and a second AAV inverted terminal repeat. The nucleic acid molecules provided can comprise a polynucleotide encoding the CLN6 polypeptide comprising an amino acid sequence at least 90% identical to amino acid sequence of SEQ ID NO: 1. In addition, the nucleic acid molecules can comprise an AAV9 genome comprising a nucleic acid sequence at least 90% identical to the nucleic acid sequence of SEQ ID NO: 4, at least 95% identical to nucleic acid sequence of SEQ ID NO: 4 or at least 98% identical to the nucleic acid sequence of SEQ ID NO: 4. Any of the nucleic acid molecules provided optionally further comprise an SV40 intron, and/or a BGH poly-A sequence.

“Packaging” refers to a series of intracellular events that result in the assembly and encapsidation of an AAV particle. The term “production” refers to the process of producing the rAAV (the infectious, encapsulated rAAV particles) by the packing cells.

AAV “rep” and “cap” genes refer to polynucleotide sequences encoding replication and encapsidation proteins, respectively, of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses may encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

The rAAV genomes provided herein lack AAV rep and cap DNA. AAV DNA in the rAAV genomes (e.g., ITRs) contemplated herein may be from any AAV serotype suitable for deriving a recombinant virus including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV rh.74 and AAV-B 1. As noted above, the nucleotide sequences of the genomes of various AAV serotypes are known in the art. rAAV with capsid mutations, are also contemplated. See, for example, Marsic et al., Molecular Therapy, 22(11): 1900-1909 (2014). Modified capsids herein are also contemplated and include capsids having various post-translational modifications such as glycosylation and deamidation. Deamidation of asparagine or glutamine side chains resulting in conversion of asparagine residues to aspartic acid or isoaspartic acid residues, and conversion of glutamine to glutamic acid or isoglutamic acid is contemplated in rAAV capsids provided herein. See, for example, Giles et al., Molecular Therapy, 26(12): 2848-2862 (2018). Modified capsids herein are also contemplated to comprise targeting sequences directing the rAAV to the affected tissues and organs requiring treatment.

DNA plasmids provided herein comprise rAAV genomes described herein. The DNA plasmids may be transferred to cells permissible for infection with a helper virus of AAV (e.g., adenovirus, E1-deleted adenovirus or herpesvirus) for assembly of the rAAV genome into infectious viral particles with AAV9 capsid proteins. Techniques to produce rAAV, in which an rAAV genome to be packaged, rep and cap genes, and helper virus functions are provided to a cell are standard in the art. Production of rAAV particles requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs. Production of pseudotyped rAAV is disclosed in, for example, WO 01/83692 which is incorporated by reference herein in its entirety. In various embodiments, AAV capsid proteins may be modified to enhance delivery of the recombinant rAAV. Modifications to capsid proteins are generally known in the art. See, for example, US 2005/0053922 and US 2009/0202490, the disclosures of which are incorporated by reference herein in their entirety.

A method of generating a packaging cell is to create a cell line that stably expresses all the necessary components for rAAV production. For example, a plasmid (or multiple plasmids) comprising a rAAV genome lacking AAV rep and cap genes, AAV rep and cap genes separate from the rAAV genome, and a selectable marker, such as a neomycin resistance gene, may be integrated into the genome of a cell. rAAV genomes may be introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line may then be infected with a helper virus such as adenovirus. The advantages of this method are that the cells are selectable and are suitable for large-scale production of rAAV. Other non-limiting examples of suitable methods employ adenovirus or baculovirus rather than plasmids to introduce rAAV genomes and/or rep and cap genes into packaging cells.

General principles of rAAV particle production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV particle production.

Further provided herein are packaging cells that produce infectious rAAV particles. In one embodiment packaging cells may be stably transformed cancer cells such as HeLa cells, 293 cells and PerC.6 cells (a cognate 293 line). In another embodiment, packaging cells may be cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Also provided herein are rAAV (e.g., infectious encapsidated rAAV particles) comprising a rAAV genome of the disclosure. The genomes of the rAAV lack AAV rep and cap DNA, that is, there is no AAV rep or cap DNA between the ITRs of the genomes of the rAAV. The rAAV genome can be a self-complementary (sc) genome. A rAAV with a sc genome is referred to herein as a scAAV. The rAAV genome can be a single-stranded (ss) genome. A rAAV with a single-stranded genome is referred to herein as an ssAAV.

An exemplary rAAV provided herein is the scAAV named “scAAV9.CB.CLN6.” The scAAV9.CB.CLN6 scAAV contains a scAAV genome comprising a human CLN6 cDNA under the control of a hybrid chicken β-Actin (CB) promoter (SEQ ID NO: 3). The scAAV genome also comprises a SV40 Intron (upstream of human CLN6 cDNA) and Bovine Growth Hormone polyadenylation (BGH Poly A) terminator sequence (downstream of human CLN6 cDNA). The sequence of this scAAV9.CB.CLN6 gene cassette is set out in SEQ ID NO: 4. The scAAV genone is packaged in an AAV9 capsid and includes AAV2 ITRs (one ITR upstream of the CB promoter and the other ITR downstream of the BGH Poly A terminator sequence).

The rAAV may be purified by methods standard in the art such as by column chromatography or cesium chloride gradients. Methods for purifying rAAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

Compositions comprising a gene therapy vector are also provided. Compositions comprise a gene therapy vector encoding a CLN6 polypeptide. Compositions may include two or more gene therapy vectors encoding different polypeptides of interest. In some embodiments, the gene therapy vector is rAAV. In some embodiments, the rAAV is scAAV or ssAAV.

Compositions provided herein comprise a gene therapy vector and a pharmaceutically acceptable excipient or excipients. In some embodiments, the gene therapy vector is rAAV. Acceptable excipients are nontoxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgI/mL, an osmolality by vapor-pressure osmometry of about 322 mOsm/kg water, an osmolarity of about 273 mOsm/L, an absolute viscosity of about 2.3cp at 20° C. and about 1.5cp at 37° C., and a specific gravity of about 1.164 at 37° C. Exemplary compositions comprise about 20 to 40% non-ionic, low-osmolar compound or about 25% to about 35% non-ionic, low-osmolar compound. An exemplary composition comprises scAAV or rAAV viral particles formulated in 20 mM Tris (pH8.0), 1 mM MgCl₂, 200 mM NaCl, 0.001% poloxamer 188 and about 25% to about 35% non-ionic, low-osmolar compound. Another exemplary composition comprises scAAV formulated in and 1×PBS and 0.001% Pluronic F68.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include about 1×10¹¹, about 1×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.5×10¹³, about 4×10¹³, about 4.5×10¹³, about 5×10¹³, about 6×10¹³, about 1×10¹⁴, about 2×10¹⁴, about 3×10¹⁴, about 4×10¹⁴ about 5×10¹⁴, about 1×10¹⁵, to about 1×10¹⁶, or more total viral genomes. Dosages of about 1×10¹¹ to about 1×10¹⁵ vg, about 1×10¹² to about 1×10¹⁵ vg, about 1×10¹² to about 1×10¹⁴ vg, about 1×10¹³ to about 6×10¹⁴ vg, and about 6×10¹³ to about 1.0×10¹⁴ vg are also contemplated. One dose exemplified herein is 6×10¹³ vg. Another dose exemplified herein is 1.5×10¹³.

Methods of transducing target cells (including, but not limited to, cells of the eye, such as photoreceptors, retinal cells, retinal ganglion cells (RGCs), retinal pigment epithelial (RPE) cells, bipolar cells, horizontal cells, amacrine cells, and Müller glia; cell of the nervous system, nerve or glial cells) with rAAV are provided. The cells of the nervous system include neurons such as lower motor neurons, microglial cells, oligodendrocytes, astrocytes, Schwann cells or combinations thereof.

The term “transduction” is used to refer to the administration/delivery of the CLN6 polynucleotide to a target cell either in vivo or in vitro, via a replication-deficient rAAV of the disclosure resulting in expression of a functional polypeptide by the recipient cell. Transduction of cells with rAAV of the disclosure results in sustained expression of polypeptide or RNA encoded by the rAAV. The present disclosure thus provides methods of administering/delivering to a subject rAAV encoding a CLN6 polypeptide by an intrathecal, intracerebroventricular, intravitreal, intraocular, subretinal, intraparechymal, or intravenous route, or any combination thereof. In some embodiments, the administration is intracerebroventricular. In some embodiments, the administration is intraocular, intravitreal, or subretinal. In some embodiments the administration is a combination of intrathecal, intracerebroventricular, intraparechymal, or intravenous and intravitreal, intraocular, or subretinal. Intrathecal delivery refers to delivery into the space under the arachnoid membrane of the brain or spinal cord. In some embodiments, intrathecal administration is via intracisternalinjection or lumbar injection.

Methods provided herein include transducing target cells (including, but not limited to, retinal cells, nerve and/or glial cells) with one or more rAAV described herein. In some embodiments, the rAAV viral particle comprising a polynucleotide encoding a CLN6 polypeptide is administered or delivered the brain, spinal cord, and/or eye of a patient. In some embodiments, the polynucleotide is delivered to the eye, such as the retina. In some embodiments, the polynucleotide is delivered to brain. Areas of the brain contemplated for delivery include, but are not limited to, the motor cortex, visual cortex, cerebellum, cerebral ventricles and the brain stem. In some embodiments, the polynucleotide is delivered to the spinal cord. In some embodiments, the polynucleotide is delivered to cells of the retina, such as photoreceptors, retinal ganglion cells (RGCs), retinal pigment epithelial (RPE) cells, bipolar cells, horizontal cells, amacrine cells, and Müller glia. In some embodiments, the polynucleotide is delivered to a neuron such as a lower motor neuron. The polynucleotide may be delivered to nerve and glial cells. The glial cell is a microglial cell, an oligodendrocyte or an astrocyte. In some embodiments, the polynucleotide is delivered to a Schwann cell.

In some embodiments of methods provided herein, the patient is held in the Trendelenberg position (head down position) after administration of the rAAV (e.g., for about 5, about 10, about 15 or about 20 minutes). For example, the patient may be tilted in the head down position at about 1 degree to about 30 degrees, about 15 to about 30 degrees, about 30 to about 60 degrees, about 60 to about 90 degrees, or about 90 to about 180 degrees).

In any of the methods provided, the compositions may be administered to a subject, including but not limited to, a human patient, at an early age. For example, the compositions provided herein may be administered to young adults, children, or infants in need thereof. In some embodiments, the compositions provided herein may be administered to a subject under 20 years old, under 15 years old, under 10 years old, under 5 years old, under 1 year old, under 6 months old, or under 1 month old. In some embodiments, the compositions provided herein may be administered to a subject in need thereof at birth. For example, the compositions may be administered to a subject that is previously identified as carrying a CLN6 mutation at fetus stage.

The terms “effective amount”, “effective dose” or “therapeutically effective amount,” as used herein, refer to a sufficient amount of an agent or a composition being administered which will reduce or ameliorate to some extent one or more of the symptoms of the disease or condition being treated; for example a reduction and/or alleviation of one or more signs, symptoms, or causes of a disease, or any other desired alteration of a biological system. An appropriate “effective” amount may be determined using techniques, such as a dose escalation study, in individual cases.

The methods provided herein comprise the step of administering an effective dose, or effective multiple doses, of a composition comprising a gene therapy vector (e.g., rAAV) provided herein to a subject (e.g., an animal including, but not limited to, a human patient) in need thereof. If the dose is administered prior to development of Batten disease, the administration is prophylactic. If the dose is administered after the development of Batten disease, the administration is therapeutic. An effective dose is a dose that alleviates (eliminates or reduces) at least one symptom associated with the disease, that slows or prevents progression of the disease, that diminishes the extent of disease, that results in remission (partial or total) of disease, and/or that prolongs survival. In comparison to the subject before treatment or in comparison to an untreated subject, methods provided herein result in stabilization, reduced progression, or improvement in one or more of the scales that are used to evaluate progression and/or improvement in Batten disease, e.g. the Unified Batten Disease Rating System (UBDRS) or the Hamburg Motor and Language Scale. The UBDRS assessment scales (as described in Marshall et al., Neurology. 2005 65(2):275-279) [including the UBDRS physical assessment scale, the UBDRS seizure assessment scale, the UBDRS behavioral assessment scale, the UBDRS capability assessment scale, the UBDRS sequence of symptom onset, and the UBDRS Clinical Global Impressions (CGI)]; the Pediatric Quality of Life Scale (PEDSQOL) scale, motor function, language function, cognitive function, and survival. In comparison to the subject before treatment or in comparison to an untreated subject, methods provided herein may result in one or more of the following: reduced or slowed degeneration of photoreceptors; reduced or slowed retinal degeneration increased number of retinal photoreceptors compared to an untreated subject; reduced or slowed lysosomal accumulation of autofluorescent storage material, reduced or slowed lysosomal accumulation of ATP Synthase Subunit C, reduced or slowed glial activation (astrocytes and/or microglia) activation; reduced or slowed astrocytosis; and showed a reduction or delay in brain volume loss measured by MRI.

Combination therapies are also provided. Combination as used herein includes either simultaneous treatment or sequential treatment. Combinations of methods described herein with standard medical treatments are specifically contemplated.

Mutations in CLN6 related to Batten disease may be tested with genetic testing. Methods of genetic testing and sequence analysis are known to those skilled in the art (Genetic Testing Registry: Ceroid lipofuscinosis neuronal 6.). In some embodiments, genetic testing may be performed on children, young adults or infants to allow early stage intervention with treatment provided herein. In some embodiments, genetic testing may be performed at fetus stage. While delivery to a subject in need thereof after birth is contemplated, intrauterine delivery to a fetus is also contemplated.

### EXAMPLES

While the following examples describe specific embodiments, it is understood that variations and modifications will occur to those skilled in the art. Accordingly, only such limitations as appear in the claims should be placed on the invention.

### Example 1

### Production of scAAV9.CB.CLN6

A human CLN6 cDNA clone was obtained from Origene, Rockville, Md. hCLN6 cDNA was further subcloned into an AAV9 genome under the hybrid chicken β-Actin promoter (CB) and tested in vitro and in vivo. A self-complementary adeno-associated virus (scAAV) serotype 9 viral genome comprising the human CLN6 (hCLN6) gene under control of the chicken-β-actin (CB) hybrid promoter was generated. A schematic of the plasmid construct showing the CLN6 cDNA inserted between AAV2 ITRs is provided in FIG. 1. The plasmid construct also includes the CP promoter, a simian virus 40 (SV40) chimeric intron and a Bovine Growth Hormone (BGH) polyadenylation signal (BGH PolyA).

scAAV9.CB.CLN6 was produced under cGMP conditions by transient triple-plasmid transfection procedures using a double-stranded AAV2-ITR-based CB-CLN6 vector, with a plasmid encoding Rep2Cap9 sequence as previously described (Gao et al., J. Virol., 78: 6381-6388 (2004)) along with an adenoviral helper plasmid pHelper™ (Stratagene, Santa Clara, Calif.) in HEK293 cells(36). The purity and titer of the vector was assessed by 4-12% sodium dodecyl sulfate-acrylamide gel electrophoresis and silver staining and qPCR analysis. After cloning, transgene expression was verified in vitro in HEK293 cells as well as in vivo via in utero ICV electroporation at embryonic day 15.5. This analysis confirmed neuronal targeting and expression of the human CLN6 protein in vivo.

### Example 2

### scAAV9.CB.CLN6 Lateral Ventricle Injection Preserves Retinal Photoreceptors

CLN6 Batten disease mouse model was used to test the effectiveness of scAAV9.CB.CLN6 for retinal preservation. Wild type and homozygous Cln6-mutant mice (Cln6^{nclf}) on C57BL/6J backgrounds were used for all studies and were housed under identical conditions. Mice received a single intracerebroventricular (ICV) injection of either PBS (n=18) or scAAV9.CB.CLN6 (n=18) (5×10¹⁰ vg/animal in 4 μL volume) at postnatal day one (P1, the day after birth) following hypothermia sedation. Animals were monitored continuously until fully recovered from sedation and daily thereafter. Subretinal injection: scAAV9.CB.CLN6 was injected at 3 (n=11) and 6 months (n=10), AAV-GFP (n=10) or PBS (n=10) injection was used as control. Eyeballs were collected at 3-, 6- and 9-month time points. Retinal cryostat sections were processed for histology and immunofluorescence.

ICV delivery of scAAV9.CB.CLN6 at P1 significantly rescued photoreceptors (8-10 layers) at both 6- and 9-month time points, compared with 0-3 layers in PBS treated eyes. The preservation of photoreceptors at the central part of the eye is more evidence than peripheral part. It is noted that degeneration rate was uneven in these mice with nasal and dorsal part of the retina degenerating slower than the temporal and ventral part of the retina. Cln6 antibody staining showed positive c1n6 in RGCs, RPE and ONL, mainly in the central part of the retina around optic nerve head. Müller glia and some inner retinal cells were also stained positively. The Subretinal injection of scAAV9.CB.CLN6 did not rescue photoreceptors, similar to AAV-GFP and PBS treated retinas. However, strong cln6 antibody staining was detected in ONL and RPE layer, not in RGCs. In addition, T cell infiltration was frequently observed in mice received subretinal injection.

### Example 3

### scAAV9.CB.CLN6 Vision Study

### High Level Study Design:

Study 1: Assessment of intracerebroventricular (ICV) injection to target visual deficits in Cln6^{nclf} mice. Animals were injected with either PBS or scAAV9.CB.CLN6 at P1 via ICV injection and assessed for functional visual acuity, pathology in the retina, and pathology in the visual processing centers of the brain at 3, 6, and 9 months of age.

Study 2: Comparison of ICV and subretinal (SR) injection to target visual deficits in Cln6^{nclf} mice. Mice were injected with either PBS, scAAV9.CB.CLN6, or scAAV9.CCB.GFP via SR injections at either 3 or 6 months of age, with retinal pathology assessed 3 months post-injection. 6 and 9 month tissues were compared to age-matched ICV injected tissues from Study 1.

Study 1 Detailed Study Design:

Injection: Postnatal day 1 (P1) WT and Cln6^{nclf} mice were injected with either 2.5e10 vg scAAV9.CB.CLN6 or PBS via intracerebroventricular injection of the lateral ventricle. All injections consisted of a volume of 2 μl.

| | 3M | | | | | | 6M | | | | | | 9M | | | | | |
|---|---|---|---|---|---|---|---|---|---|---|---|---|---|---|---|---|---|---|
| | | | | | Cln6^{nclf} | | | | | | Cln6^{nclf} | | | | | | Cln6^{nclf} | |
| | | | | | ScAAV9. | | | | | | ScAAV9. | | | | | | ScAAV9. | |
| | WT | | Cln6^{nclf} | | CB.CLN6 | | WT | | Cln6^{nclf} | | CB.CLN6 | | WT | | Cln6^{nclf} | | CB.CLN6 | |
| | M | F | M | F | M | F | M | F | M | F | M | F | M | F | M | F | M | F |
| Visual | 11 | 13 | 13 | 16 | 17 | 11 | 6 | 5 | 10 | 12 | 14 | 8 | 3 | 3 | 6 | 4 | 11 | 6 |
| Assessment | | | | | | | | | | | | | | | | | | |
| Brain | 6 | 6 | 6 | 6 | 6 | 6 | 3 | 3 | 3 | 3 | 3 | 3 | 3 | 3 | 3 | 3 | 3 | 3 |
| Pathology* | | | | | | | | | | | | | | | | | | |
| Retinal | 3 | 3 | 3 | 3 | 3 | 3 | 3 | 3 | 3 | 3 | 3 | 3 | 3 | 3 | 3 | 3 | 3 | 3 |
| Pathology | | | | | | | | | | | | | | | | | | |

Functional Visual Assessment: Animals were assessed for visual acuity at 3, 6, and 9 months of age using an OptoMotry™ system by Cerebral Mechanics. The system consists of four computer monitors that surround the mouse and project a rotating sine-wave grating of varying contrasts to the animal. The user monitors the animal using an overhead camera, noting whether the animal tracks the grating with reflexive head and neck movements. Based on user responses on animal tracking, the OptoMotry software calculates the spatial visual acuity of the animal (cycles/degree).

Tissue Collection: Animals were CO₂ euthanized and cardiac-perfused at 3, 6, or 9 months of age. Eyes were removed, cornea punctured, and fixed in modified Davidson's solution for 24 hours. Brains were removed, placed into a 1 mm brain block, and bisected into two hemispheres. 1 hemisphere was placed in 4% PFA for 24 hours, while the other hemisphere was bisected once more and pieces separately flash frozen. Additional collections included: blood, serum, spinal cord, heart, liver, spleen, and kidney for fixed and frozen tissue banking.

Study 2 Detailed Study Design:

Injection and Tissue Collection: 3 or 6 month Cln6^{nclf} mice were anesthetized and a small incision was made in the sclera to allow for the placement of a pulled glass capillary between the sclera and the retinal layers. Animals were injected with 2.5e10 vg scAAV9.CB.CLN6 in one eye, and either 2.5e10 vg scAAV9.CB.GFP or PBS injected into the contralateral eye. All injections consisted of a volume of 2 μl. Animals were CO₂ euthanized and cardiac-perfused 3 months post-injection. Eyes were removed, cornea punctured, and fixed in modified Davidson's solution for 24 hours. Brains were removed, placed into a 1 mm brain block, and bisected into two hemispheres. 1 hemisphere was placed in 4% PFA for 24 hours, while the other hemisphere was bisected once more and pieces separately flash frozen.

Number of Cln6^{nclf} Eyes Collected

| 3M SR Injection, 6M Collection | | | 6M SR Injection, 9M Collection | | |
|---|---|---|---|---|---|
| PBS | scAAV9.CB.CLN6 | scAAV9.CB.GFP | PBS | scAAV9.CB.CLN6 | scAAV9.CB.GFP |
| 5 | 10 | 5 | 5 | 10 | 5 |

Study 1 Results:

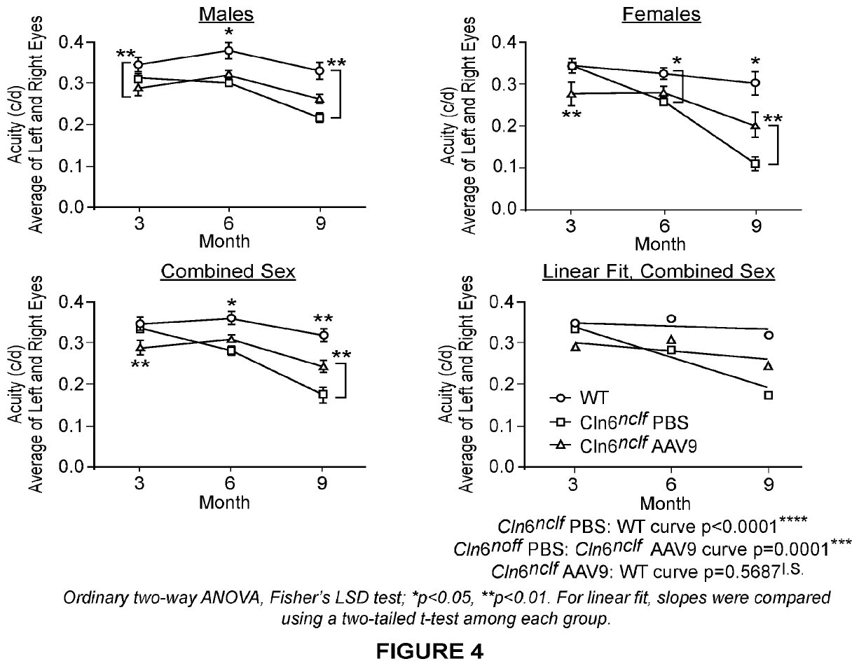

Functional Visual Assessment: Cln6^{nclf} mice had lower visual acuity compared to WT counterparts beginning at 6 months of age. To assess whether the preservation of retinal and other visual pathway neurons rescued visual function, animals were tested for visual ability using optokinetic tracking from 3 to 9 months of age (OptoMotry, Cerebral Mechanics). Briefly, animals were individually placed on a platform surrounded by four monitors arranged in a square, and a gradient of varying contrasts rotated around the mice in either a clockwise or counterclockwise fashion. To assess visual acuity, an experimenter blinded to genotype and treatment status observed the animal and determined whether the animal was tracking the gradient. Beginning at 6 months of age, untreated Cln6^{nclf} animals of both sexes declined in spatial visual acuity (cycles/degree), with deficits progressing until the last time point at 9 months of age (FIG. 4). Importantly, untreated Cln6^{nclf} mice, particularly females, had a significantly steeper decline in vision over the three month period when compared to either scAAV9.CB.CLN6 treated Cln6^{nclf} or wild type control mice (comparison of slope). Indeed, visual performance in scAAV9.CB.CLN6 treated Cln6^{nclf} animals declined in a similar manner to wild type animals (comparison of slope), maintaining visual acuity between wild type and untreated Cln6^{nclf} by 9 months of age.

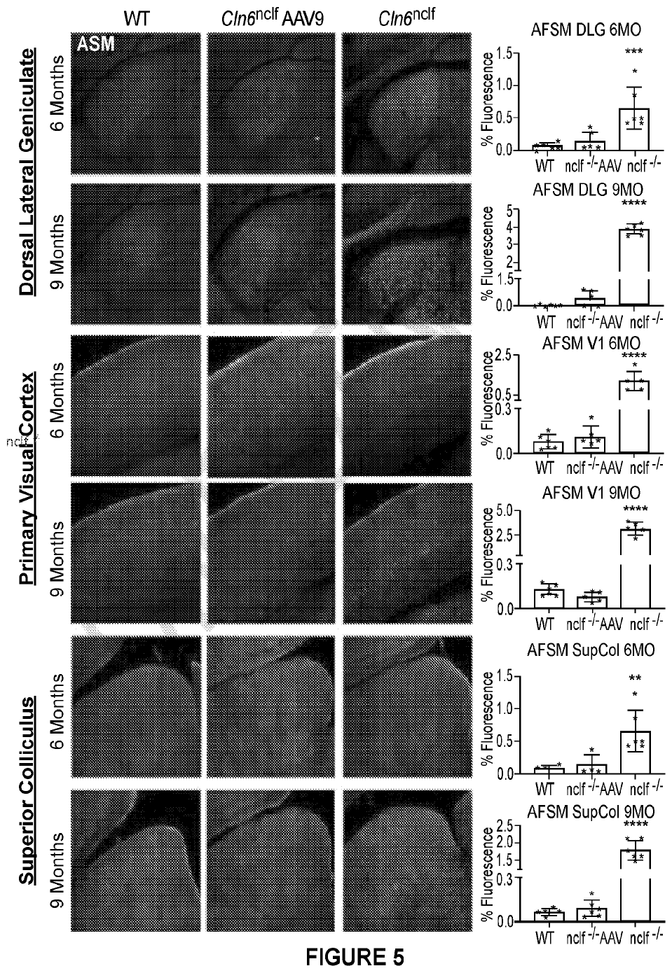

Brain Pathology, Visual Centers: A single, ICV injection of scAAV9.CB.CLN6 at P1 prevented autofluorescent storage material accumulation (ASM) in Cln6^{nclf} mice, in three areas of the brain associated with the visual system. FIG. 5 demostrates strong autofluorescent storage material (ASM) accumulation in Cln6^{nclf} animals in dorsal lateral geniculate, primary visual cortex, and superior collicus, with neonatal scAAV9.CB.CLN6 treatment preventing this characteristic accumulation at all time points examined.

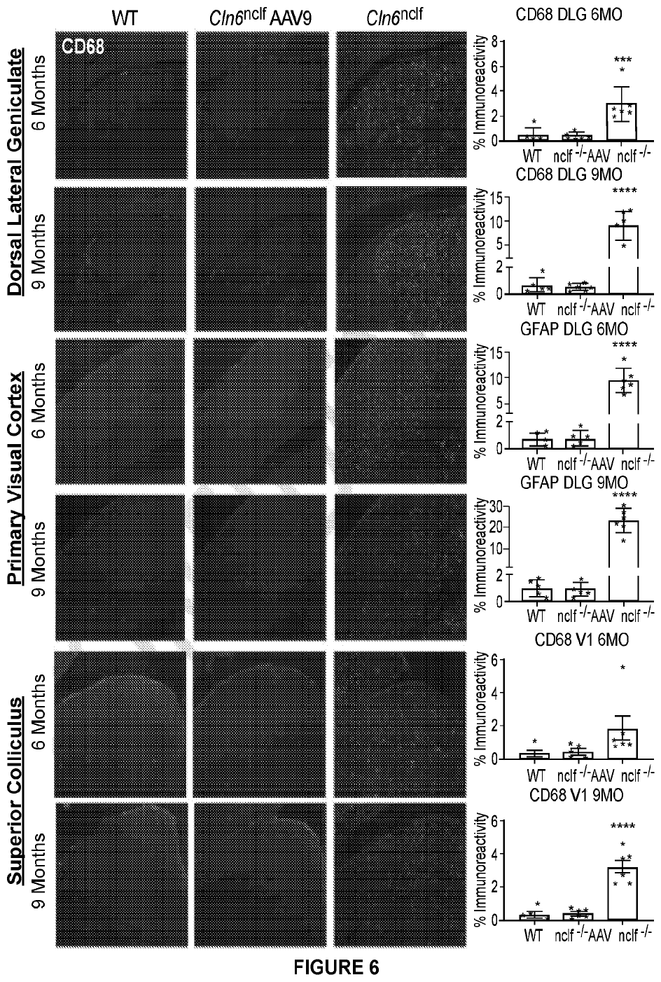

Similar to ASM accumulation, Cln6^{nclf} animals presented with robust astrocyte (GFAP⁺) and microglial (CD68⁺) immunoreactivity in all visual centers examined. A single, ICV injection of scAAV9.CB.CLN6 at P1 prevented microglial activation (CD68) in Cln6^{nclf} mice in the dorsal lateral geniculate, primary visual cortex, and superior collicus at all time points examined (FIG. 6).

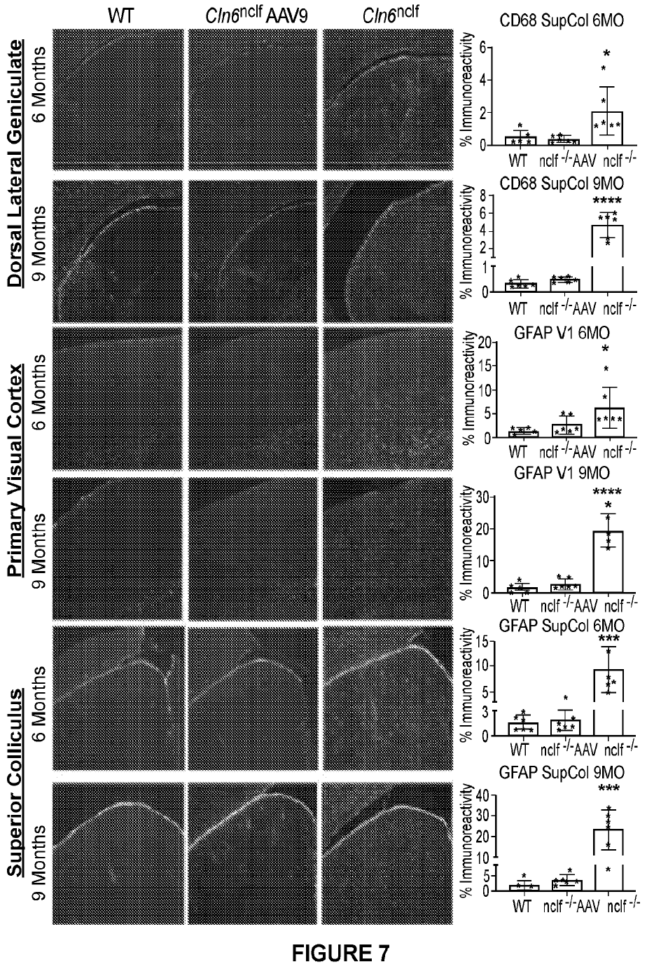

A single, ICV injection of scAAV9.CB.CLN6 at P1 prevented astrocyte reactivity (GFAP) in Cln6^{nclf} mice, in three areas of the brain associated with the visual system (FIG. 7).

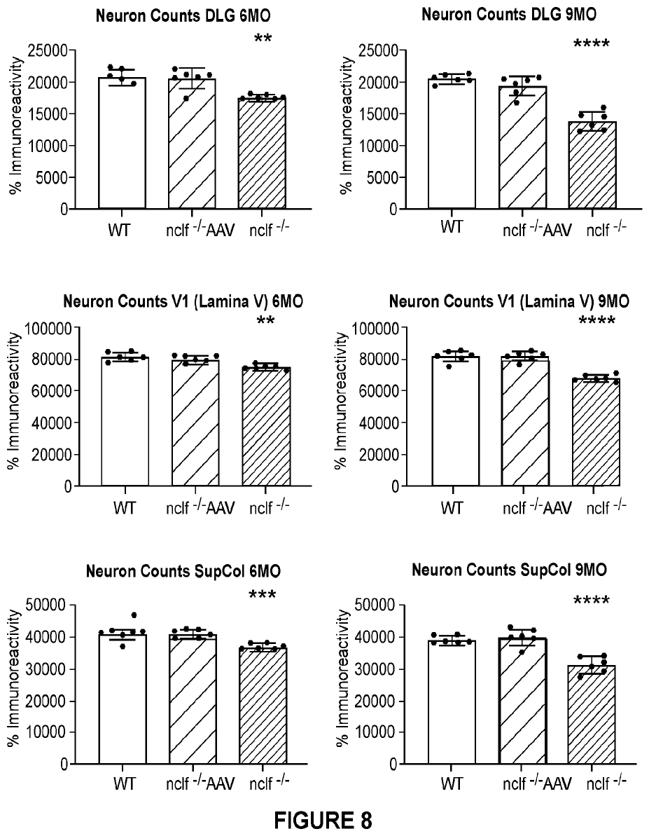

A single, ICV injection of scAAV9.CB.CLN6 at P1 prevented neuronal loss in Cln6^{nclf} mice, in three areas of the brain associated with the visual system, at all time points examined (FIG. 8).

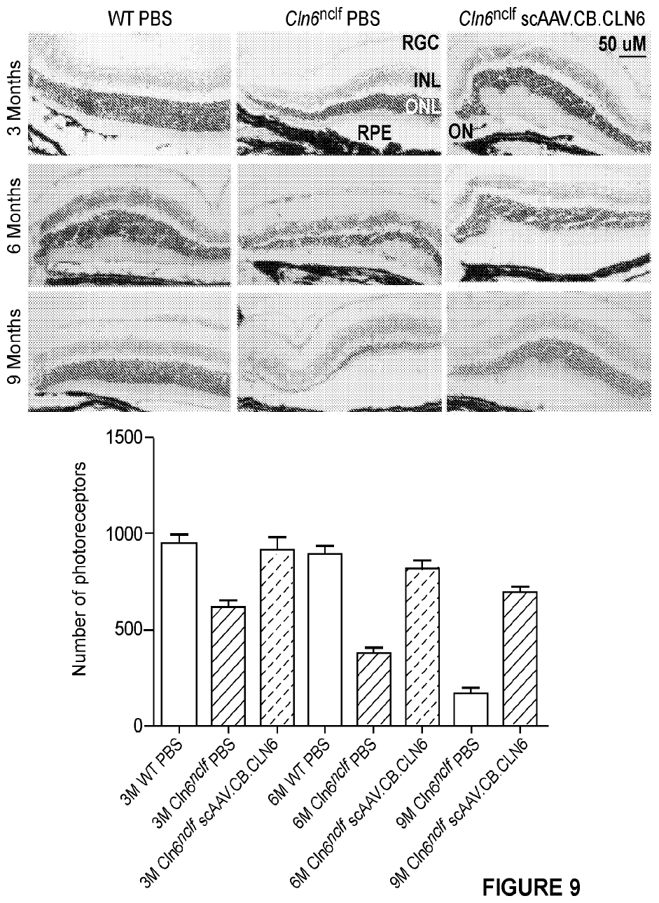

Retinal Pathology: As neuronal integrity was preserved in the visual processing centers of the brain in AAV9-treated Cln6^{nclf} mice, neuronal integrity in the retina was also exampled to see if this pathology had also been prevented. Untreated Cln6^{nclf} mice progressively lost photoreceptors from 3 to 9 months of age, with particularly prominent degeneration in the outer nuclear layer (arrows) and in the retinal periphery (FIG. 5). A single, ICV injection of scAAV9.CB.CLN6 at P1 retained the retinal ganglion, inner nuclear, and outer nuclear layers in Cln6^{nclf} mice. Untreated Cln6^{nclf} mice began retinal thinning of these layers at 3 months of age. Additionally, AAV9 treatment maintained a high number of photoreceptors until 9 months of age. (FIG. 9). Wild type animals presented with 10 layers of photoreceptors at each time point examined, with scAAV9.CB.CLN6 preserving these layers in the central retina of Cln6^{nclf} mice until 9 months of age. However, photoreceptors in the peripheral retina continued to be lost following scAAV9.CB.CLN6 treatment with age, notably at 9 months of age.

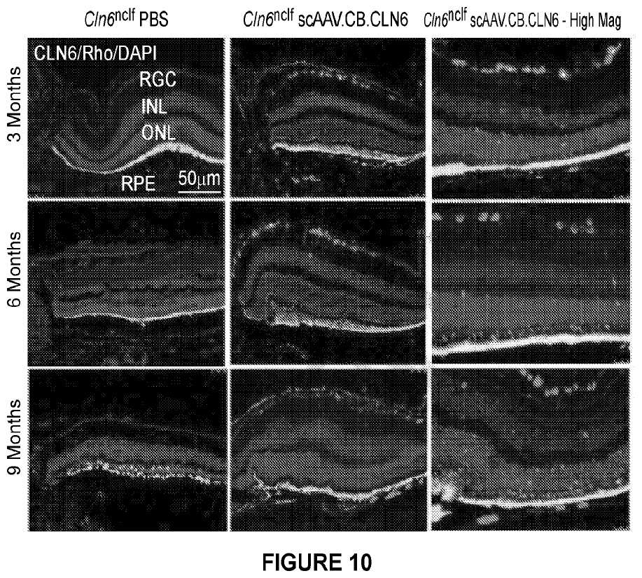

Given the unexpected degree of photoreceptor protection in Cln6^{nclf} mice treated with an ICV injection of scAAV9.CB.CLN6, it was investigated whether human CLN6 was present in the retina of treated mice. To characterize the expression patterns of ICV-delivered scAAV9.CB.CLN6 in the retina, endogenous mouse Cln6 transcript was visualized with a modified in situ technique (RNAscope) and transgenic human CLN6 (hCLN6) protein with immunolabeling (a lack of commercial antibodies targeting mouse Cln6 precluded dual immunolabeling). Robust expression of the transgene was observed throughout the retina. While endogenous mCln6 transcript was present primarily in the inner and outer nuclear layers, with lower level expression in retinal ganglion cells (RGCs) and the retinal pigmented epithelium (FIG. 10), hCLN6 was expressed widely throughout the RGC layer, inner nuclear layer, outer nuclear layer, retinal pigmented epithelium, and choroid, with particularly robust expression in RGCs. When co-labeled with a marker for Willer glia (glutamine synthetase (GS⁺)) and PKCa, a marker for bipolar cells, hCLN6 immunolabeling did not colocalize with these cells (data not shown for PKC staining), consistent with previous reports of predominantly neuronal expression following subretinal delivery of AAV9 (Watanabe et al. PLoS One 8: e54146). Taken together, a single ICV injection of scAAV9.CB.CLN6 transduces the retina and significantly preserves photoreceptors in Cln6^{nclf} mice, despite its somewhat ectopic retinal expression patterns.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

All documents referred to in this application are hereby incorporated by reference in their entirety.

| SEQUENCES | |
|---|---|
| CLN6 Polypeptide Sequence | |
| SEQ ID NO: 1 | |
| Met Glu Ala Thr Arg Arg Arg Gln His Leu Gly Ala Thr Gly Gly Pro | |
| Gly Ala Gln Leu Gly Ala Ser Phe Leu Gln Ala Arg His Gly Ser Val | |
| Ser Ala Asp Glu Ala Ala Arg Thr Ala Pro Phe His Leu Asp Leu Trp | |
| Phe Tyr Phe Thr Leu Gln Asn Trp Val Leu Asp Phe Gly Arg Pro Ile | |
| Ala Met Leu Val Phe Pro Leu Glu Trp Phe Pro Leu Asn Lys Pro Ser | |
| Val Gly Asp Tyr Phe His Met Ala Tyr Asn Val Ile Thr Pro Phe Leu | |
| Leu Leu Lys Leu Ile Glu Arg Ser Pro Arg Thr Leu Pro Arg Ser Ile | |
| Thr Tyr Val Ser Ile Ile Ile Phe Ile Met Gly Ala Ser Ile His Leu | |
| Val Gly Asp Ser Val Asn His Arg Leu Leu Phe Ser Gly Tyr Gln His | |
| His Leu Ser Val Arg Glu Asn Pro Ile Ile Lys Asn Leu Lys Pro Glu | |
| Thr Leu Ile Asp Ser Phe Glu Leu Leu Tyr Tyr Tyr Asp Glu Tyr Leu | |
| Gly His Cys Met Trp Tyr Ile Pro Phe Phe Leu Ile Leu Phe Met Tyr | |
| Phe Ser Gly Cys Phe Thr Ala Ser Lys Ala Glu Ser Leu Ile Pro Gly | |
| Pro Ala Leu Leu Leu Val Ala Pro Ser Gly Leu Tyr Tyr Trp Tyr Leu | |
| Val Thr Glu Gly Gln Ile Phe Ile Leu Phe Ile Phe Thr Phe Phe Ala | |
| Met Leu Ala Leu Val Leu His Gln Lys Arg Lys Arg Leu Phe Leu Asp | |
| Ser Asn Gly Leu Phe Leu Phe Ser Ser Phe Ala Leu Thr Leu Leu Leu | |
| Val Ala Leu Trp Val Ala Trp Leu Trp Asn Asp Pro Val Leu Arg Lys | |
| Lys Tyr Pro Gly Val Ile Tyr Val Pro Glu Pro Trp Ala Phe Tyr Thr | |
| Leu His Val Ser Ser Arg His | |
| CLN6 polynucleotide sequence | |
| SEQ ID NO: 2 | |
| atggaggcga cgcggaggcg gcagcacctg ggagcgacgg gcggcccagg cgcgcagctg | 60 |
| ggcgcctcct tcctgcaggc caggcatggc tctgtgagcg ctgatgaggc tgcccgcacg | 120 |
| gctcccttcc acctcgacct ctggttctac ttcacactgc agaactgggt tctggacttt | 180 |
| gggcgtccca ttgccatgct ggtattccct ctcgagtggt ttccactcaa caagcccagt | 240 |
| gttggggact acttccacat ggcctacaac gtcatcacgc cctttctctt gctcaagctc | 300 |
| atcgagcggt ccccccgcac cctgccacgc tccatcacgt acgtgagcat catcatcttc | 360 |
| atcatgggtg ccagcatcca cctggtgggt gactctgtca accaccgcct gctcttcagt | 420 |
| ggctaccagc accacctgtc tgtccgtgag aaccccatca tcaagaatct caagccggag | 480 |
| acgctgatcg actcctttga gctgctctac tattatgatg agtacctggg tcactgcatg | 540 |
| tggtacatcc ccttcttcct catcctcttc atgtacttca gcggctgctt tactgcctct | 600 |
| aaagctgaga gcttgattcc agggcctgcc ctgctcctgg tggcacccag tggcctgtac | 660 |
| tactggtacc tggtcaccga gggccagatc ttcatcctct tcatcttcac cttcttcgcc | 720 |
| atgctggccc tcgtcctgca ccagaagcgc aagcgcctct tcctggacag caacggcctc | 780 |
| ttcctcttct cctccttcgc actgaccctc ttgcttgtgg cgctctgggt cgcctggctg | 840 |
| tggaatgacc ctgttctcag gaagaagtac ccgggtgtca tctacgtccc tgagccctgg | 900 |
| gctttctaca cccttcacgt cagcagtcgg cactga | 936 |
| hybrid chicken B-Actin promoter | |
| SEQ ID NO: 3 | |
| ccacgttctg cttcactctc cccatctccc ccccctcccc acccccaatt ttgtatttat | 60 |
| ttatttttta attattttgt gcagcgatgg gggcgggggg gggggggggg cgcgcgccag | 120 |
| gcggggcggg gcggggcgag gggcggggcg gggcgaggcg gagaggtgcg gcggcagcca | 180 |
| atcagagcgg cgcgctccga aagtttcctt ttatggcgag gcggcggcgg cggcggccct | 240 |
| ataaaaagcg aagcgcgcgg cgggcgggag | 270 |
| Gene Cassette | |
| SEQ ID NO: 4 | |
| ctgcgcgctc gctcgctcac tgaggccgcc cgggcaaagc ccgggcgtcg ggcgaccttt | 60 |
| ggtcgcccgg cctcagtgag cgagcgagcg cgcagagagg gagtggaatt cacgcgtgga | 120 |
| tctgaattca attcacgcgt ggtacctctg gtcgttacat aacttacggt aaatggcccg | 180 |
| cctggctgac cgcccaacga cccccgccca ttgacgtcaa taatgacgta tgttcccata | 240 |
| gtaacgccaa tagggacttt ccattgacgt caatgggtgg agtatttacg gtaaactgcc | 300 |
| cacttggcag tacatcaagt gtatcatatg ccaagtacgc cccctattga cgtcaatgac | 360 |
| ggtaaatggc ccgcctggca ttatgcccag tacatgacct tatgggactt tcctacttgg | 420 |
| cagtacatct actcgaggcc acgttctgct tcactctccc catctccccc ccctccccac | 480 |
| ccccaatttt gtatttattt attttttaat tattttgtgc agcgatgggg gcgggggggg | 540 |
| ggggggggcg cgcgccaggc ggggcggggc ggggcgaggg gcggggcggg gcgaggcgga | 600 |
| gaggtgcggc ggcagccaat cagagcggcg cgctccgaaa gtttcctttt atggcgaggc | 660 |
| ggcggcggcg gcggccctat aaaaagcgaa gcgcgcggcg ggcgggagcg ggatcagcca | 720 |
| ccgcggtggc ggcctagagt cgacgaggaa ctgaaaaacc agaaagttaa ctggtaagtt | 780 |
| tagtcttttt gtcttttatt tcaggtcccg gatccggtgg tggtgcaaat caaagaactg | 840 |
| ctcctcagtg gatgttgcct ttacttctag gcctgtacgg aagtgttact tctgctctaa | 900 |
| aagctgcgga attgtacccg cggccgatcc accggtctta agggccgagg cggccagatc | 960 |
| tttcgaagat atcggcgccg ctagcgcggc cgcatggagg cgacgcggag gcggcagcac | 1020 |
| ctgggagcga cgggcggccc aggcgcgcag ctgggcgcct ccttcctgca ggccaggcat | 1080 |
| ggctctgtga gcgctgatga ggctgcccgc acggctccct tccacctcga cctctggttc | 1140 |
| tacttcacac tgcagaactg ggttctggac tttgggcgtc ccattgccat gctggtattc | 1200 |
| cctctcgagt ggtttccact caacaagccc agtgttgggg actacttcca catggcctac | 1260 |
| aacgtcatca cgccctttct cttgctcaag ctcatcgagc ggtccccccg caccctgcca | 1320 |
| cgctccatca cgtacgtgag catcatcatc ttcatcatgg gtgccagcat ccacctggtg | 1380 |
| ggtgactctg tcaaccaccg cctgctcttc agtggctacc agcaccacct gtctgtccgt | 1440 |
| gagaacccca tcatcaagaa tctcaagccg gagacgctga tcgactcctt tgagctgctc | 1500 |
| tactattatg atgagtacct gggtcactgc atgtggtaca tccccttctt cctcatcctc | 1560 |
| ttcatgtact tcagcggctg ctttactgcc tctaaagctg agagcttgat tccagggcct | 1620 |
| gccctgctcc tggtggcacc cagtggcctg tactactggt acctggtcac cgagggccag | 1680 |
| atcttcatcc tcttcatctt caccttcttc gccatgctgg ccctcgtcct gcaccagaag | 1740 |
| cgcaagcgcc tcttcctgga cagcaacggc ctcttcctct tctcctcctt cgcactgacc | 1800 |
| ctcttgcttg tggcgctctg ggtcgcctgg ctgtggaatg accctgttct caggaagaag | 1860 |
| tacccgggtg tcatctacgt ccctgagccc tgggctttct acacccttca cgtcagcagt | 1920 |
| cggcactgag tctctagaaa gcttatcgat accgtcgact agagctcgct gatcagcctc | 1980 |
| gactgtgcct tctagttgcc agccatctgt tgtttgcccc tcccccgtgc cttccttgac | 2040 |
| cctggaaggt gccactccca ctgtcctttc ctaataaaat gaggaaattg catcgcattg | 2100 |
| tctgagtagg tgtcattcta ttctgggggg tggggtgggg caggacagca agggggagga | 2160 |
| ttgggaagac aatagcaggc atgctgggga gagatcgatc tgaggaaccc ctagtgatgg | 2220 |
| agttggccac tccctctctg cgcgctcgct cgctcactga ggccgggcga ccaaaggtcg | 2280 |
| cccgacgccc gggctttgcc cgggcggcct cagtgagcga gcgagcgcgc agagagggag | 2340 |
| tgg | 2343 |
| AAV.CB.CLN6 | |
| SEQ ID NO: 5 | |
| ctgattctaa cgaggaaagc acgttatacg tgctcgtcaa agcaaccata gtacgcgccc | 60 |
| tgtagcggcg cattaagcgc ggcgggtgtg gtggttacgc gcagcgtgac cgctacactt | 120 |
| gccagcgccc tagcgcccgc tcctttcgct ttcttccctt cctttctcgc cacgttcgcc | 180 |
| ggctttcccc gtcaagctct aaatcggggg ctccctttag ggttccgatt tagtgcttta | 240 |
| cggcacctcg accccaaaaa acttgattag ggtgatggtt cacgtagtgg gccatcgccc | 300 |
| tgatagacgg tttttcgccc tttgacgttg gagtccacgt tctttaatag tggactcttg | 360 |
| ttccaaactg gaacaacact caaccctatc tcggtctatt cttttgattt ataagggatt | 420 |
| ttgccgattt cggcctattg gttaaaaaat gagctgattt aacaaaaatt taacgcgaat | 480 |
| tttaacaaaa tattaacgct tacaatttaa atatttgctt atacaatctt cctgtttttg | 540 |
| gggcttttct gattatcaac cggggtacat atgattgaca tgctagtttt acgattaccg | 600 |
| ttcatcgccc tgcgcgctcg ctcgctcact gaggccgccc gggcaaagcc cgggcgtcgg | 660 |
| gcgacctttg gtcgcccggc ctcagtgagc gagcgagcgc gcagagaggg agtggaattc | 720 |
| acgcgtggat ctgaattcaa ttcacgcgtg gtacctctgg tcgttacata acttacggta | 780 |
| aatggcccgc ctggctgacc gcccaacgac ccccgcccat tgacgtcaat aatgacgtat | 840 |
| gttcccatag taacgccaat agggactttc cattgacgtc aatgggtgga gtatttacgg | 900 |
| taaactgccc acttggcagt acatcaagtg tatcatatgc caagtacgcc ccctattgac | 960 |
| gtcaatgacg gtaaatggcc cgcctggcat tatgcccagt acatgacctt atgggacttt | 1020 |
| cctacttggc agtacatcta ctcgaggcca cgttctgctt cactctcccc atctcccccc | 1080 |
| cctccccacc cccaattttg tatttattta ttttttaatt attttgtgca gcgatggggg | 1140 |
| cggggggggg gggggggcgc gcgccaggcg gggcggggcg gggcgagggg cggggcgggg | 1200 |
| cgaggcggag aggtgcggcg gcagccaatc agagcggcgc gctccgaaag tttcctttta | 1260 |
| tggcgaggcg gcggcggcgg cggccctata aaaagcgaag cgcgcggcgg gcgggagcgg | 1320 |
| gatcagccac cgcggtggcg gcctagagtc gacgaggaac tgaaaaacca gaaagttaac | 1380 |
| tggtaagttt agtctttttg tcttttattt caggtcccgg atccggtggt ggtgcaaatc | 1440 |
| aaagaactgc tcctcagtgg atgttgcctt tacttctagg cctgtacgga agtgttactt | 1500 |
| ctgctctaaa agctgcggaa ttgtacccgc ggccgatcca ccggtcttaa gggccgaggc | 1560 |
| ggccagatct ttcgaagata tcggcgccgc tagcgcggcc gcatggaggc gacgcggagg | 1620 |
| cggcagcacc tgggagcgac gggcggccca ggcgcgcagc tgggcgcctc cttcctgcag | 1680 |
| gccaggcatg gctctgtgag cgctgatgag gctgcccgca cggctccctt ccacctcgac | 1740 |
| ctctggttct acttcacact gcagaactgg gttctggact ttgggcgtcc cattgccatg | 1800 |
| ctggtattcc ctctcgagtg gtttccactc aacaagccca gtgttgggga ctacttccac | 1860 |
| atggcctaca acgtcatcac gccctttctc ttgctcaagc tcatcgagcg gtccccccgc | 1920 |
| accctgccac gctccatcac gtacgtgagc atcatcatct tcatcatggg tgccagcatc | 1980 |
| cacctggtgg gtgactctgt caaccaccgc ctgctcttca gtggctacca gcaccacctg | 2040 |
| tctgtccgtg agaaccccat catcaagaat ctcaagccgg agacgctgat cgactccttt | 2100 |
| gagctgctct actattatga tgagtacctg ggtcactgca tgtggtacat ccccttcttc | 2160 |
| ctcatcctct tcatgtactt cagcggctgc tttactgcct ctaaagctga gagcttgatt | 2220 |
| ccagggcctg ccctgctcct ggtggcaccc agtggcctgt actactggta cctggtcacc | 2280 |
| gagggccaga tcttcatcct cttcatcttc accttcttcg ccatgctggc cctcgtcctg | 2340 |
| caccagaagc gcaagcgcct cttcctggac agcaacggcc tcttcctctt ctcctccttc | 2400 |
| gcactgaccc tcttgcttgt ggcgctctgg gtcgcctggc tgtggaatga ccctgttctc | 2460 |
| aggaagaagt acccgggtgt catctacgtc cctgagccct gggctttcta cacccttcac | 2520 |
| gtcagcagtc ggcactgagt ctctagaaag cttatcgata ccgtcgacta gagctcgctg | 2580 |
| atcagcctcg actgtgcctt ctagttgcca gccatctgtt gtttgcccct cccccgtgcc | 2640 |
| ttccttgacc ctggaaggtg ccactcccac tgtcctttcc taataaaatg aggaaattgc | 2700 |
| atcgcattgt ctgagtaggt gtcattctat tctggggggt ggggtggggc aggacagcaa | 2760 |
| gggggaggat tgggaagaca atagcaggca tgctggggag agatcgatct gaggaacccc | 2820 |
| tagtgatgga gttggccact ccctctctgc gcgctcgctc gctcactgag gccgggcgac | 2880 |
| caaaggtcgc ccgacgcccg ggctttgccc gggcggcctc agtgagcgag cgagcgcgca | 2940 |
| gagagggagt ggcccccccc cccccccccc cggcgattct cttgtttgct ccagactctc | 3000 |
| aggcaatgac ctgatagcct ttgtagagac ctctcaaaaa tagctaccct ctccggcatg | 3060 |
| aatttatcag ctagaacggt tgaatatcat attgatggtg atttgactgt ctccggcctt | 3120 |
| tctcacccgt ttgaatcttt acctacacat tactcaggca ttgcatttaa aatatatgag | 3180 |
| ggttctaaaa atttttatcc ttgcgttgaa ataaaggctt ctcccgcaaa agtattacag | 3240 |
| ggtcataatg tttttggtac aaccgattta gctttatgct ctgaggcttt attgcttaat | 3300 |
| tttgctaatt ctttgccttg cctgtatgat ttattggatg ttggaatcgc ctgatgcggt | 3360 |
| attttctcct tacgcatctg tgcggtattt cacaccgcat atggtgcact ctcagtacaa | 3420 |
| tctgctctga tgccgcatag ttaagccagc cccgacaccc gccaacacta tggtgcactc | 3480 |
| tcagtacaat ctgctctgat gccgcatagt taagccagcc ccgacacccg ccaacacccg | 3540 |
| ctgacgcgcc ctgacgggct tgtctgctcc cggcatccgc ttacagacaa gctgtgaccg | 3600 |
| tctccgggag ctgcatgtgt cagaggtttt caccgtcatc accgaaacgc gcgagacgaa | 3660 |
| agggcctcgt gatacgccta tttttatagg ttaatgtcat gataataatg gtttcttaga | 3720 |
| cgtcaggtgg cacttttcgg ggaaatgtgc gcggaacccc tatttgttta tttttctaaa | 3780 |
| tacattcaaa tatgtatccg ctcatgagac aataaccctg ataaatgctt caataatatt | 3840 |
| gaaaaaggaa gagtatgagt attcaacatt tccgtgtcgc ccttattccc ttttttgcgg | 3900 |
| cattttgcct tcctgttttt gctcacccag aaacgctggt gaaagtaaaa gatgctgaag | 3960 |
| atcagttggg tgcacgagtg ggttacatcg aactggatct caacagcggt aagatccttg | 4020 |
| agagttttcg ccccgaagaa cgttttccaa tgatgagcac ttttaaagtt ctgctatgtg | 4080 |
| gcgcggtatt atcccgtatt gacgccgggc aagagcaact cggtcgccgc atacactatt | 4140 |
| ctcagaatga cttggttgag tactcaccag tcacagaaaa gcatcttacg gatggcatga | 4200 |
| cagtaagaga attatgcagt gctgccataa ccatgagtga taacactgcg gccaacttac | 4260 |
| ttctgacaac gatcggagga ccgaaggagc taaccgcttt tttgcacaac atgggggatc | 4320 |
| atgtaactcg ccttgatcgt tgggaaccgg agctgaatga agccatacca aacgacgagc | 4380 |
| gtgacaccac gatgcctgta gcaatggcaa caacgttgcg caaactatta actggcgaac | 4440 |
| tacttactct agcttcccgg caacaattaa tagactggat ggaggcggat aaagttgcag | 4500 |
| gaccacttct gcgctcggcc cttccggctg gctggtttat tgctgataaa tctggagccg | 4560 |
| gtgagcgtgg gtctcgcggt atcattgcag cactggggcc agatggtaag ccctcccgta | 4620 |
| tcgtagttat ctacacgacg gggagtcagg caactatgga tgaacgaaat agacagatcg | 4680 |
| ctgagatagg tgcctcactg attaagcatt ggtaactgtc agaccaagtt tactcatata | 4740 |
| tactttagat tgatttaaaa cttcattttt aatttaaaag gatctaggtg aagatccttt | 4800 |
| ttgataatct catgaccaaa atcccttaac gtgagttttc gttccactga gcgtcagacc | 4860 |
| ccgtagaaaa gatcaaagga tcttcttgag atcctttttt tctgcgcgta atctgctgct | 4920 |
| tgcaaacaaa aaaaccaccg ctaccagcgg tggtttgttt gccggatcaa gagctaccaa | 4980 |
| ctctttttcc gaaggtaact ggcttcagca gagcgcagat accaaatact gttcttctag | 5040 |
| tgtagccgta gttaggccac cacttcaaga actctgtagc accgcctaca tacctcgctc | 5100 |
| tgctaatcct gttaccagtg gctgctgcca gtggcgataa gtcgtgtctt accgggttgg | 5160 |
| actcaagacg atagttaccg gataaggcgc agcggtcggg ctgaacgggg ggttcgtgca | 5220 |
| cacagcccag cttggagcga acgacctaca ccgaactgag atacctacag cgtgagctat | 5280 |
| gagaaagcgc cacgcttccc gaagggagaa aggcggacag gtatccggta agcggcaggg | 5340 |
| tcggaacagg agagcgcacg agggagcttc cagggggaaa cgcctggtat ctttatagtc | 5400 |
| ctgtcgggtt tcgccacctc tgacttgagc gtcgattttt gtgatgctcg tcaggggggc | 5460 |
| ggagcctatg gaaaaacgcc agcaacgcgg cctttttacg gttcctggcc ttttgctggc | 5520 |
| cttttgctca catgttcttt cctgcgttat cccctgattc tgtggataac cgtattaccg | 5580 |
| cctttgagtg agctgatacc gctcgccgca gccgaacgac cgagcgcagc gagtcagtga | 5640 |
| gcgaggaagc ggaagagcgc ccaatacgca aaccgcctct ccccgcgcgt tggccgattc | 5700 |
| attaatgcag ctggcgtaat agcgaagagg cccgcaccga tcgcccttcc caacagttgc | 5760 |
| gcagcctgaa tggcgaatgg cgattccgtt gcaatggctg gcggtaatat tgttctggat | 5820 |
| attaccagca aggccgatag tttgagttct tctactcagg caagtgatgt tattactaat | 5880 |
| caaagaagta ttgcgacaac ggttaatttg cgtgatggac agactctttt actcggtggc | 5940 |
| ctcactgatt ataaaaacac ttctcaggat tctggcgtac cgttcctgtc taaaatccct | 6000 |
| ttaatcggcc tcctgtttag ctcccgct | 6028 |
| CMV Sequence | |
| SEQ ID NO: 6 | |
| cgttacataa cttacggtaa atggcccgcc tggctgaccg cccaacgacc cccgcccatt | 60 |
| gacgtcaata atgacgtatg ttcccatagt aacgccaata gggactttcc attgacgtca | 120 |
| atgggtggag tatttacggt aaactgccca cttggcagta catcaagtgt atcatatgcc | 180 |
| aagtacgccc cctattgacg tcaatgacgg taaatggccc gcctggcatt atgcccagta | 240 |
| catgacctta tgggactttc ctacttggca gtacatctac | 280 |
| scAAV9 sequence | |
| SEQ ID NO: 7 | |
| Met Ala Ala Asp Gly Tyr Leu Pro Asp Trp Leu Glu Asp Asn Leu Ser | |
| Glu Gly Ile Arg Glu Trp Trp Ala Leu Lys Pro Gly Ala Pro Gln Pro | |
| Lys Ala Asn Gln Gln His Gln Asp Asn Ala Arg Gly Leu Val Leu Pro | |
| Gly Tyr Lys Tyr Leu Gly Pro Gly Asn Gly Leu Asp Lys Gly Glu Pro | |
| Val Asn Ala Ala Asp Ala Ala Ala Leu Glu His Asp Lys Ala Tyr Asp | |
| Gln Gln Leu Lys Ala Gly Asp Asn Pro Tyr Leu Lys Tyr Asn His Ala | |
| Asp Ala Glu Phe Gln Glu Arg Leu Lys Glu Asp Thr Ser Phe Gly Gly | |
| Asn Leu Gly Arg Ala Val Phe Gln Ala Lys Lys Arg Leu Leu Glu Pro | |
| Leu Gly Leu Val Glu Glu Ala Ala Lys Thr Ala Pro Gly Lys Lys Arg | |
| Pro Val Glu Gln Ser Pro Gln Glu Pro Asp Ser Ser Ala Gly Ile Gly | |
| Lys Ser Gly Ala Gln Pro Ala Lys Lys Arg Leu Asn Phe Gly Gln Thr | |
| Gly Asp Thr Glu Ser Val Pro Asp Pro Gln Pro Ile Gly Glu Pro Pro | |
| Ala Ala Pro Ser Gly Val Gly Ser Leu Thr Met Ala Ser Gly Gly Gly | |
| Ala Pro Val Ala Asp Asn Asn Glu Gly Ala Asp Gly Val Gly Ser Ser | |
| Ser Gly Asn Trp His Cys Asp Ser Gln Trp Leu Gly Asp Arg Val Ile | |
| Thr Thr Ser Thr Arg Thr Trp Ala Leu Pro Thr Tyr Asn Asn His Leu | |
| Tyr Lys Gln Ile Ser Asn Ser Thr Ser Gly Gly Ser Ser Asn Asp Asn | |
| Ala Tyr Phe Gly Tyr Ser Thr Pro Trp Gly Tyr Phe Asp Phe Asn Arg | |
| Phe His Cys His Phe Ser Pro Arg Asp Trp Gln Arg Leu Ile Asn Asn | |
| Asn Trp Gly Phe Arg Pro Lys Arg Leu Asn Phe Lys Leu Phe Asn Ile | |
| Gln Val Lys Glu Val Thr Asp Asn Asn Gly Val Lys Thr Ile Ala Asn | |
| Asn Leu Thr Ser Thr Val Gln Val Phe Thr Asp Ser Asp Tyr Gln Leu | |
| Pro Tyr Val Leu Gly Ser Ala His Glu Gly Cys Leu Pro Pro Phe Pro | |
| Ala Asp Val Phe Met Ile Pro Gln Tyr Gly Tyr Leu Thr Leu Asn Asp | |
| Gly Ser Gln Ala Val Gly Arg Ser Ser Phe Tyr Cys Leu Glu Tyr Phe | |
| Pro Ser Gln Met Leu Arg Thr Gly Asn Asn Phe Gln Phe Ser Tyr Glu | |
| Phe Glu Asn Val Pro Phe His Ser Ser Tyr Ala His Ser Gln Ser Leu | |
| Asp Arg Leu Met Asn Pro Leu Ile Asp Gln Tyr Leu Tyr Tyr Leu Ser | |
| Lys Thr Ile Asn Gly Ser Gly Gln Asn Gln Gln Thr Leu Lys Phe Ser | |
| Val Ala Gly Pro Ser Asn Met Ala Val Gln Gly Arg Asn Tyr Ile Pro | |
| Gly Pro Ser Tyr Arg Gln Gln Arg Val Ser Thr Thr Val Thr Gln Asn | |
| Asn Asn Ser Glu Phe Ala Trp Pro Gly Ala Ser Ser Trp Ala Leu Asn | |
| Gly Arg Asn Ser Leu Met Asn Pro Gly Pro Ala Met Ala Ser His Lys | |
| Glu Gly Glu Asp Arg Phe Phe Pro Leu Ser Gly Ser Leu Ile Phe Gly | |
| Lys Gln Gly Thr Gly Arg Asp Asn Val Asp Ala Asp Lys Val Met Ile | |
| Thr Asn Glu Glu Glu Ile Lys Thr Thr Asn Pro Val Ala Thr Glu Ser | |
| Tyr Gly Gln Val Ala Thr Asn His Gln Ser Ala Gln Ala Gln Ala Gln | |
| Thr Gly Trp Val Gln Asn Gln Gly Ile Leu Pro Gly Met Val Trp Gln | |
| Asp Arg Asp Val Tyr Leu Gln Gly Pro Ile Trp Ala Lys Ile Pro His | |
| Thr Asp Gly Asn Phe His Pro Ser Pro Leu Met Gly Gly Phe Gly Met | |
| Lys His Pro Pro Pro Gln Ile Leu Ile Lys Asn Thr Pro Val Pro Ala | |
| Asp Pro Pro Thr Ala Phe Asn Lys Asp Lys Leu Asn Ser Phe Ile Thr | |
| Gln Tyr Ser Thr Gly Gln Val Ser Val Glu Ile Glu Trp Glu Leu Gln | |
| Lys Glu Asn Ser Lys Arg Trp Asn Pro Glu Ile Gln Tyr Thr Ser Asn | |
| Tyr Tyr Lys Ser Asn Asn Val Glu Phe Ala Val Asn Thr Glu Gly Val | |
| Tyr Ser Glu Pro Arg Pro Ile Gly Thr Arg Tyr Leu Thr Arg Asn Leu | |
| AAV2 3′ ITR | |
| SEQ ID NO: 8 | |
| aggaacccct agtgatggag ttggccactc cctctctgcg cgctcgctcg ctcactgagg | 60 |
| ccgggcgacc aaaggtcgcc cgacgcccgg gctttgcccg ggcggcctca gtgagcgagc | 120 |
| gagcgcgcag agagggagtg g | 141 |
| AAV2 5′ ITR | |
| SEQ ID NO: 9 | |
| ctgcgcgctc gctcgctcac tgaggccgcc cgggcaaagc ccgggcgtcg ggcgaccttt | 60 |
| ggtcgcccgg cctcagtgag cgagcgagcg cgcagagagg gagtgg | 106 |
| BGH Poly A | |
| SEQ ID NO: 10 | |
| cctcgactgt gccttctagt tgccagccat ctgttgtttg cccctccccc gtgccttcct | 60 |
| tgaccctgga aggtgccact cccactgtcc tttcctaata aaatgaggaa attgcatcgc | 120 |
| attgtctgag taggtgtcat tctattc | 147 |
| SV40 intron | |
| SEQ ID NO: 11 | |
| gtaagtttag tctttttgtc ttttatttca ggtcccggat ccggtggtgg tgcaaatcaa | 60 |
| Page | |

What is claimed is:

1. A method of preserving photoreceptors in an individual with Batten disease in need thereof, comprising administering to the individual a pharmaceutical composition comprising a therapeutically effective amount of a gene therapy vector encoding a CLN6 polypeptide.

2. A method of inhibiting retinal degeneration in an individual with Batten disease in need thereof, comprising administering to the individual a pharmaceutical composition comprising a therapeutically effective amount of a gene therapy vector encoding a CLN6 polypeptide.

3. A method of treating the visual effects of an individual with Batten disease in need thereof, comprising administering to the individual a pharmaceutical composition comprising a therapeutically effective amount of a gene therapy vector encoding a CLN6 polypeptide.

4. The method of any one of claims 1-3, wherein the CLN6 polypeptide is at least 90% identical to a polypeptide of SEQ ID NO:1.

5. The method of any one of claims 1-3, wherein the gene therapy vector is a viral vector.

6. The method of claim 5, wherein the viral vector is an adeno-associated virus (AAV), an adenovirus, a retrovirus, a pox virus, a lentivirus, an adenovirus, a vaccinia virus, or a herpes simplex virus.

7. The method of claim 6, wherein the viral vector is an AAV.

8. The method of claim 7, wherein the AAV is selected from the group consisting of an AAV1, an AAV2, an AAV3, an AAV4, an AAV5, an AAV6, an AAV7, an AAV8, an AAV9, an AAVrhS, an AAVrh10 vector, an AAVrh33, an AAVrh34, an AAVrh74, an AAV Anc80, an AAVPHP.B, and an AAV-DJ.

9. The method of claim 7 or 8, wherein the AAV is a recombinant AAV9 (rAAV9) comprising an rAAV9 genome comprising, in 5′ to 3′ order: a first inverted repeat, a chicken beta actin (CB) promoter comprising the nucleic acid sequence of SEQ ID NO: 3, a polynucleotide encoding a Ceroid lipofuscinosis neuron protein 6 (CLN6) polypeptide comprising the amino acid sequence 90% identical to SEQ ID NO:1, and a second inverted repeat.

10. The method of claim 9, wherein the rAAV9 genome further comprises a cytomegalovirus (CMV) enhancer.

11. The method of claim 9 or 10, wherein the rAAV9 genome further comprises a SV40 intron.

12. The method of any one of claims 9-11, wherein the rAAV9 genome further comprises a bovine growth hormone polyadenylation poly A sequence.

13. The method of any one of claims 9-12, wherein the rAAV9 genome is a single-stranded genome or a self-complementary genome.

14. The method of any one of claims 9-13, wherein the rAAV9 genome is a self-complementary genome comprising in 5′ to 3′ order: a first AAV inverted terminal repeat, a CMV enhancer, a CB promoter comprising the nucleotide sequence of SEQ ID NO: 3, an SV40 intron, a polynucleotide encoding the CLN6 polypeptide of SEQ ID NO: 1 and a second AAV inverted terminal repeat.

15. The method of any one of claims 9-13, wherein the rAAV9 genome is a self-complementary genome comprising: a first AAV inverted terminal repeat, a CB promoter comprising the sequence of SEQ ID NO: 3, a polynucleotide encoding the CLN6 polypeptide of SEQ ID NO: 1, a bovine growth hormone polyadenylation poly A sequence and a second AAV inverted terminal repeat.

16. The method of any one of claims 9-13, wherein the rAAV9 genome is a self-complementary genome comprising: a first AAV inverted terminal repeat, a CMV enhancer, a CB promoter comprising the sequence of SEQ ID NO: 3, an SV40 intron, a polynucleotide encoding the CLN6 polypeptide of SEQ ID NO: 1, a bovine growth hormone polyadenylation poly A sequence and a second AAV inverted terminal repeat.

17. The method of any one of claims 9-16, wherein the AAV inverted terminal repeats are AAV2 inverted terminal repeats.

18. The method of any one of claims 1-17, wherein the pharmaceutical composition is administered intracerebroventricularly, intrathecally, intraperenchymally, intravenously, subretinally, intraocularly, intravitreally, or a combination thereof.

19. The method of claim 2, wherein the pharmaceutical composition is administered intracerebroventricularly.

20. The method of any one of claims 1-19, wherein about 1×10⁸ vg to about 1×10¹⁵ vg of the rAAV viral particle is administered per gram body weight of the individual.

21. The method of any one of claims 1-20, wherein symptoms of visual failure are prevented or ameliorated.

22. The method of claim any one of claims 1-21, wherein photoreceptor cells in the central retina of the individual are substantially preserved.

23. The method of any one of claims 1-22, wherein the individual comprises a retina comprising at least 4 layers of photoreceptor cells 6 months after the treatment.

24. The method of any one of claims 1-22, wherein the individual comprises a retina comprising at least 8 layers of photoreceptor cells 6 months after the treatment.

25. The method of any one of claims 1-22, wherein the individual comprises a retina comprising at least 4 layers of photoreceptor cells 9 months after the treatment.

26. The method of any one of claims 1-22, wherein the individual comprises a retina comprising at least 8 layers of photoreceptor cells 9 months after the treatment.

27. The method of any one of claims 1-26, wherein the individual is a less than 10 years old.

28. The method of claim 27, wherein the individual is less than 1 year old.

29. The method of any one of claims 1-28, wherein the individual comprises a CLN6 gene comprising a mutation related to Batten disease.

30. The method of any one of claims 1-29, the method further comprises detecting a mutation related to Batten disease in a CLN6 gene of the individual.

31. The method of any one of claims 1-30, wherein the pharmaceutical composition further comprises a pharmaceutically acceptable excipient, carrier, or diluent.

32. The method of claim 31, wherein the excipient comprises a non-ionic, low-osmolar compound, a buffer, a polymer, a salt, or a combination thereof.

33. The method of claim 32, wherein the non-ionic, low-osmolar contrast agent is selected from the group consisting of iobitridol, iohexol, iomeprol, iopamidol, iopentol, iopromide, ioversol, ioxilan, and combinations thereof.

34. A composition for preserving photoreceptors in an individual with Batten disease in need thereof, wherein the composition comprises a therapeutically effective amount of a gene therapy vector encoding a CLN6 polypeptide.

35. A composition for inhibiting retinal degeneration in an individual with Batten disease in need thereof, wherein the composition comprises a therapeutically effective amount of a gene therapy vector encoding a CLN6 polypeptide.

36. A composition for treating the visual effects of an individual with Batten disease in need thereof, wherein the composition comprises a therapeutically effective amount of a gene therapy vector encoding a CLN6 polypeptide.

37. The composition of any one of claims 34-36, wherein the CLN6 polypeptide is at least 90% identical to a polypeptide of SEQ ID NO:1.

38. The composition of any one of claims 34-36, wherein the gene therapy vector is a viral vector.

39. The composition of claim 38, wherein the viral vector is an adeno-associated virus (AAV), an adenovirus, a retrovirus, a pox virus, a lentivirus, an adenovirus, a vaccinia virus, or a herpes simplex virus.

40. The composition of claim 39, wherein the viral vector is an AAV.

41. The composition of claim 40, wherein the AAV is selected from the group consisting of an AAV1, an AAV2, an AAV3, an AAV4, an AAV5, an AAV6, an AAV7, an AAV8, an AAV9, an AAVrhS, an AAVrh10 vector, an AAVrh33, an AAVrh34, an AAVrh74, an AAV Anc80, an AAVPHP.B, and an AAV-DJ.

42. The composition of claim 41, wherein the AAV is a recombinant AAV9 (rAAV9) comprising an rAAV9 genome comprising, in 5′ to 3′ order: a first inverted repeat, a chicken beta actin (CB) promoter comprising the nucleic acid sequence of SEQ ID NO: 3, a polynucleotide encoding a Ceroid lipofuscinosis neuron protein 6 (CLN6) polypeptide comprising the amino acid sequence 90% identical to SEQ ID NO:1, and a second inverted repeat.

43. The composition of claim 42, wherein the rAAV9 genome further comprises a cytomegalovirus (CMV) enhancer.

44. The composition of claim 42 or 43, wherein the rAAV9 genome further comprises a SV40 intron.

45. The method of any one of claims 42-44, wherein the rAAV9 genome further comprises a bovine growth hormone polyadenylation poly A sequence.

46. The composition of any one of claims 42-45, wherein the rAAV9 genome is a single-stranded genome or a self-complementary genome.

47. The composition of claim 42, wherein the rAAV9 genome is a self-complementary genome comprising in 5′ to 3′ order: a first AAV inverted terminal repeat, a CMV enhancer, a CB promoter comprising the nucleotide sequence of SEQ ID NO: 3, an SV40 intron, a polynucleotide encoding the CLN6 polypeptide of SEQ ID NO: 1 and a second AAV inverted terminal repeat.

48. The composition of claim 42, wherein the rAAV9 genome is a self-complementary genome comprising: a first AAV inverted terminal repeat, a CB promoter comprising the sequence of SEQ ID NO: 3, a polynucleotide encoding the CLN6 polypeptide of SEQ ID NO: 1, a bovine growth hormone polyadenylation poly A sequence and a second AAV inverted terminal repeat.

49. The composition of claim 42, wherein the rAAV9 genome is a self-complementary genome comprising: a first AAV inverted terminal repeat, a CMV enhancer, a CB promoter comprising the sequence of SEQ ID NO: 3, an SV40 intron, a polynucleotide encoding the CLN6 polypeptide of SEQ ID NO: 1, a bovine growth hormone polyadenylation poly A sequence and a second AAV inverted terminal repeat.

50. The composition of any one of claims 42-50, wherein the AAV inverted terminal repeats are AAV2 inverted terminal repeats.

51. The composition of any one of claims 35-50, wherein the composition is formulated for administered intracerebroventricularly, intrathecally, intraperenchymally, intravenously, subretinally, intraocularly, intravitreally, or a combination thereof.

52. The composition of claim 51, wherein the pharmaceutical composition is administered intracerebroventricularly.

53. The composition of any one of claims 34-52, wherein the composition comprises about 1×10⁸ vg to about 1×10¹⁵ vg of the rAAV viral particle per gram body weight of the individual.

54. The composition of any one of claims 34-53, wherein administration of the composition prevents or ameliorates the symptoms of visual failure are prevented or ameliorated.

55. The composition of any one of claims 35-54, wherein administration of the composition substantially preserves the photoreceptor cells in the central retina of the individual.

56. The composition of any one of claims 34-55, wherein the individual comprises a retina comprising at least 4 layers of photoreceptor cells 6 months after administration of the composition.

57. The composition of any one of claims 34-55, wherein the individual comprises a retina comprising at least 8 layers of photoreceptor cells 6 months after administration of the composition.

58. The composition of any one of claims 34-55, wherein the individual comprises a retina comprising at least 4 layers of photoreceptor cells 9 months after administration of the composition.

59. The composition of any one of claims 34-55, wherein the individual comprises a retina comprising at least 8 layers of photoreceptor cells 9 months after administration of the composition.

60. The composition of any one of claims 34-59, wherein the individual is a less than 10 years old.

61. The composition of claim 60, wherein the individual is less than 1 year old.

62. The composition of any one of claims 34-61, wherein the individual comprises a CLN6 gene comprising a mutation related to Batten disease.

63. The composition of any one of claims 34-62, wherein the composition further comprises a pharmaceutically acceptable excipient, carrier, or diluent.

64. The composition of claim 63, wherein the excipient comprises a non-ionic, low-osmolar compound, a buffer, a polymer, a salt, or a combination thereof.

65. The composition of claim 64, wherein the non-ionic, low-osmolar contrast agent is selected from the group consisting of iobitridol, iohexol, iomeprol, iopamidol, iopentol, iopromide, ioversol, ioxilan, and combinations thereof.

66. Use of a therapeutically effective amount of a gene therapy vector encoding a CLN6 polypeptide for the preparation of a medicament for preserving photoreceptors in an individual with Batten disease in need thereof.

67. Use of a therapeutically effective amount of a gene therapy vector encoding a CLN6 polypeptide for the preparation of a medicament for inhibiting retinal degeneration in an individual with Batten disease in need thereof.

68. Use of a therapeutically effective amount of a gene therapy vector encoding a CLN6 polypeptide for the preparation of a medicament for treating the visual effects of an individual with Batten disease in need thereof.

69. The use of any one of claims 66-68, wherein the CLN6 polypeptide is at least 90% identical to a polypeptide of SEQ ID NO:1.

70. The use of any one of claims 66-69, wherein the gene therapy vector is a viral vector.

71. The use of claim 70, wherein the viral vector is an adeno-associated virus (AAV), an adenovirus, a retrovirus, a pox virus, a lentivirus, an adenovirus, a vaccinia virus, or a herpes simplex virus.

72. The use of claim 71, wherein the viral vector is an AAV.

73. The use of claim 72, wherein the AAV is selected from the group consisting of an AAV1, an AAV2, an AAV3, an AAV4, an AAV5, an AAV6, an AAV7, an AAV8, an AAV9, an AAVrhS, an AAVrh10 vector, an AAVrh33, an AAVrh34, an AAVrh74, an AAV Anc80, an AAVPHP.B, and an AAV-DJ.

74. The use of claim 72 or 73, wherein the AAV is a recombinant AAV9 (rAAV9) comprising an rAAV9 genome comprising, in 5′ to 3′ order: a first inverted repeat, a chicken beta actin (CB) promoter comprising the nucleic acid sequence of SEQ ID NO: 3, a polynucleotide encoding a Ceroid lipofuscinosis neuron protein 6 (CLN6) polypeptide comprising the amino acid sequence 90% identical to SEQ ID NO:1, and a second inverted repeat.

75. The use of claim 74, wherein the rAAV9 genome further comprises a cytomegalovirus (CMV) enhancer.

76. The use of claim 74 or 75, wherein the rAAV9 genome further comprises a SV40 intron.

77. The use of any one of claims 74-76, wherein the rAAV9 genome further comprises a bovine growth hormone polyadenylation poly A sequence.

78. The use of any one of claims 74-77, wherein the rAAV9 genome is a single-stranded genome or a self-complementary genome.

79. The use of any one of claims 74-78, wherein the rAAV9 genome is a self-complementary genome comprising in 5′ to 3′ order: a first AAV inverted terminal repeat, a CMV enhancer, a CB promoter comprising the nucleotide sequence of SEQ ID NO: 3, an SV40 intron, a polynucleotide encoding the CLN6 polypeptide of SEQ ID NO: 1 and a second AAV inverted terminal repeat.

80. The use of any one of claims 74-78, wherein the rAAV9 genome is a self-complementary genome comprising: a first AAV inverted terminal repeat, a CB promoter comprising the sequence of SEQ ID NO: 3, a polynucleotide encoding the CLN6 polypeptide of SEQ ID NO: 1, a bovine growth hormone polyadenylation poly A sequence and a second AAV inverted terminal repeat.

81. The use of any one of claims 74-78, wherein the rAAV9 genome is a self-complementary genome comprising: a first AAV inverted terminal repeat, a CMV enhancer, a CB promoter comprising the sequence of SEQ ID NO: 3, an SV40 intron, a polynucleotide encoding the CLN6 polypeptide of SEQ ID NO: 1, a bovine growth hormone polyadenylation poly A sequence and a second AAV inverted terminal repeat.

82. The use of any one of claims 74-81, wherein the AAV inverted terminal repeats are AAV2 inverted terminal repeats.

83. The use of any one of claims 66-82, wherein the medicament is formulated for administration intracerebroventricularly, intrathecally, intraperenchymally, intravenously, subretinally, intraocularly, intravitreally, or a combination thereof.

84. The use of claim 83, wherein the pharmaceutical composition is administered intracerebroventricularly.

85. The use of any one of claims 66-84, wherein the medicament comprises about 1×10⁸ vg to about 1×10¹⁵ vg of the rAAV viral particle per gram body weight of the individual.

86. The use of any one of claims 66-85, wherein administration of medicament prevents or ameliorates the symptoms of visual failure in the individual.

87. The use of any one of claims 66-85, wherein administration of the medicament substantially preserves the photoreceptor cells in the central retina of the individual.

88. The use of any one of claims 67-87, wherein the individual comprises a retina comprising at least 4 layers of photoreceptor cells 6 months after administration of the medicament.

89. The use of any one of claims 67-87, wherein the individual comprises a retina comprising at least 8 layers of photoreceptor cells 6 months after administration of the medicament.

90. The use of any one of claims 67-87, wherein the individual comprises a retina comprising at least 4 layers of photoreceptor cells 9 months after administration of the medicament.

91. The use of any one of claims 67-87, wherein the individual comprises a retina comprising at least 8 layers of photoreceptor cells 9 months after administration of the medicament.

92. The use of any one of claims 66-91, wherein the individual is a less than 10 years old.

93. The use of claim 92, wherein the individual is less than 1 year old.

94. The use of any one of claims 66-93, wherein the individual comprises a CLN6 gene comprising a mutation related to Batten disease.

95. The use of any one of claims 66-94, the method further comprises detecting a mutation related to Batten disease in a CLN6 gene of the individual.

96. The use of any one of claims 66-95, wherein the medicament further comprises a pharmaceutically acceptable excipient, carrier, or diluent.

97. The use of claim 96, wherein the excipient comprises a non-ionic, low-osmolar compound, a buffer, a polymer, a salt, or a combination thereof.

98. The use of claim 97, wherein the non-ionic, low-osmolar contrast agent is selected from the group consisting of iobitridol, iohexol, iomeprol, iopamidol, iopentol, iopromide, ioversol, ioxilan, and combinations thereof.
